(19) United States
(12) Patent Application Publication
(10) Pub. No.: US 2024/0173406 A1
(43) Pub. Date: May 30, 2024

(54) MATERIALS AND METHODS FOR GENERATING ANTIGEN-SPECIFIC T CELLS AND TREATING DISEASES

(71) Applicant: NUTCRACKER THERAPEUTICS, INC., Emeryville, CA (US)

(72) Inventors: Samuel Deutsch, Walnut Creek, CA (US); Ole Haabeth, Alameda, CA (US)

(21) Appl. No.: 18/284,701

(22) PCT Filed: Apr. 1, 2022
(86) PCT No.: PCT/US22/23125

§ 371 Date: Sep. 28, 2023

Related U.S. Application Data
(60) Provisional application No. 63/170,221, filed on Apr. 2, 2021.

(51) Int. Cl. A61K 39/00; C12N 5/0783

(52) U.S. Cl. CPC A61K 39/4611; A61K 39/0011; A61K 39/46433; A61K 39/464499; C12N 5/0636; A61K 2039/53; A61K 2039/55555

(57) ABSTRACT

The disclosure provides materials in the form of nanoparticles containing in vitro-transcribed mRNAs encoding antigenic peptides and personalized medicine methods for treating or preventing diseases such as cancer, an autoimmune disease, an infectious disease, or inflammation, wherein cells of a subject receiving prophylactic or therapeutic treatment are brought into contact with the nanoparticles ex vivo, and the subject's cells process the mRNA. expressing and presenting the encoded product to activate the subject's own T cells. thereby mounting an immune response to the diseased cells. such as cancer cells.

### CROSS-REFERENCE TO RELATED APPLICATIONS

This application claims the priority benefit under 35 U.S.C. § 119(3) of U.S. Provisional Patent Application No. 63/170,221, filed Apr. 2, 2021, which is incorporated herein by reference in its entirety.

### INCORPORATION BY REFERENCE OF MATERIAL SUBMITTED ELECTRONICALLY

This application contains, as a separate part of the disclosure, a Sequence Listing in computer-readable form which is incorporated by reference in its entirety and identified as follows: Filename: 56579_Seqlisting.txt; Size 6,544 bytes; Created: Mar. 31, 2022.

### BACKGROUND

In recent years, immunotherapy for the treatment of solid cancer has emerged as a promising therapeutic alternative. In cancer immunotherapy, the immune system is either passively or actively exploited to target and kill cancer cells. Immunotherapy offers target specificity that abrogates off-target toxicities while still inducing highly potent anti-cancer responses. By targeting tumor cells or their microenvironment, passive immunotherapy can enhance endogenous anti-tumor immune response by overcoming suppression and inhibit tumor cell growth. In active immunotherapy, immune cells are stimulated and instructed to actively fight cancer and although more challenging, this approach is extremely promising. Active immunotherapy is highly dependent on efficient stimulation of antigen-specific immune cells, such as killer T cells and antibody-producing B cells. In adoptive T cell transfer, isolated autologous tumor-specific T cells are expanded ex vivo and, after sufficient stimulation, are reinfused into the cancer patient, where these cells are expected to elicit potent anti-tumor responses (1). Adoptive cell therapy (ACT), especially T cell-based, has seen limited success in causing tumor regression, as revealed by the percentage of patients responding to treatment. In addition, checkpoint inhibitors further underscore the potential of the T cell compartment in the treatment of cancer. Not all patients respond to these treatments, however, and many challenges remain.

Obstacles at several different levels contribute to preventing the success of T cell ACT in solid tumors. One difficulty is target selection. The target antigen chosen for T cell immunotherapy can be a known tumor antigen or can be a neoantigen formed by unique mutations found in one specific tumor, or even unknown, as in the case of tumor-infiltrating lymphocyte (TIL) therapy.

### SUMMARY

The disclosure provides materials and methods for generating an antigen-specific T cell for treatment of disease. Various implementations of the materials and methods are described below, and the materials and methods, including and excluding the additional implementations enumerated below, in any combination (provided these combinations are not inconsistent), may overcome these shortcomings and achieve the benefits described herein.

The disclosure provides materials and methods for the generation of antigenic materials in the form of delivery vehicles encapsulating an mRNA to form an mRNA nanoparticle. The encapsulated mRNA encodes, at a minimum, an antigenic peptide or antigenic polypeptide useful in eliciting a therapeutic immune response to treat disease such as cancer (e.g., tumors), inflammation, infectious disease, and autoimmune diseases. Cancer is the disease characterized by uncontrolled cell growth due to loss of cell cycle control in at least one cell type. Some cancerous cells may form solid masses in the form of tumors. Tumors may be benign or malignant. Benign tumors may not result in a cancer, but malignant tumors are a form of cancer, and may be considered to be comprised of cancerous cells. Inflammation is the condition characterized by redness, swelling, pain, and increased temperature resulting from a physiological process to protect against injury, disease, irritation or invasion by a foreign body or organism. Infectious diseases are diseases caused by foreign organisms that come in contact with, or invade, a subject. Infectious agents may be a bacterium, a fungus, or a virus. Autoimmune diseases are diseases characterized by a body's immune system improperly protecting the body against self antigens. The methods exploit self-antigens in the form of antigenic peptides that are not delivered directly to immune cells but are encoded by mRNAs packaged in delivery vehicles encapsulating the mRNAs to form mRNA nanoparticles (e.g., mRNA lipid nanoparticles). The mRNA nanoparticles may partially or completely enclose the cargo of the nanoparticle, such as at least one mRNA according to this disclosure. An antigen-presenting cell of the subject being treated is transfected with an mRNA encoding at least one antigenic peptide and that transfected APC presents the antigenic peptide to T cells of that same subject, ex vivo, e.g., in vitro. Activation and expansion of T cells may be promoted by presentation-enhancing sequences and effector compositions. The result is a personalized medicine approach to the immunotherapy to treat a variety of diseases exemplified by the treatment of cancers such as solid tumor cancers.

One aspect of this disclosure is drawn to a method comprising: characterizing polynucleotides from a diseased tissue relative to a control; identifying candidate mRNAs encoding polypeptides associated with the diseased tissue from the characterized polynucleotides; encapsulating the mRNAs in with at least one delivery vehicle molecule to form at least one mRNA nanoparticle; introducing the at least one mRNA nanoparticle, wherein the mRNAs encode the polypeptides, to peripheral blood leukocytes or sentinel lymph node leukocytes of the subject; contacting the peripheral blood leukocytes or sentinel lymph node leukocytes comprising mRNAs with T cells of the subject; and obtaining at least one antigen-specific T cell. By delivering mRNA nanoparticles comprising delivery vehicle molecules encapsulating the mRNAs to leukocytes, the mRNAs of the mRNA nanoparticles may be processed and translated to yield encoded peptides. Peptides as used herein refers to both polypeptide fragments and full-length polypeptides. In some implementations, the subject is a human. In some implementations, the disease is cancer, an infectious disease, an autoimmune disease or inflammation.

In some implementations, the subject has cancer. In some implementations, the cancer is Acute Lymphoblastic Leukemia (ALL), Acute Myeloid Leukemia (AML), Adrenocortical Carcinoma, Kaposi Sarcoma, Lymphoma, Anal Cancer, Astrocytomas, Atypical Teratoid/Rhabdoid Tumor, Basal Cell Carcinoma of the Skin, Bile Duct Cancer, Bladder Cancer, Bone Cancer, Brain Cancer, Tumors, Breast Cancer, Bronchial Tumors, Carcinoid Tumor, Cardiac (Heart) Tumors, Medulloblastoma, Germ Cell Tumor, Cervical Cancer , Cholangiocarcinoma, Chordoma, Chronic Lymphocytic Leukemia (CLL), Chronic Myelogenous Leukemia (CML), Colorectal Cancer, Craniopharyngioma, Cutaneous T-Cell Lymphoma, Ductal Carcinoma In Situ (DCIS), Embryonal Tumor, Ependymoma, Esophageal Cancer, Esthesioneuroblastoma (Head and Neck Cancer), Ewing Sarcoma (Bone Cancer), Extracranial Germ Cell Tumor, Eye Cancer, Intraocular Melanoma, Retinoblastoma, Fallopian Tube Cancer, Fibrous Histiocytoma of Bone, Osteosarcoma, Gallbladder Cancer, Gastric (Stomach) Cancer, Gastrointestinal Carcinoid Tumor, Gastrointestinal Stromal Tumors (GIST) (Soft Tissue Sarcoma), Extragonadal Germ Cell Tumors, Ovarian Germ Cell Tumors, Testicular Cancer, Gestational Trophoblastic Disease, Hairy Cell Leukemia, Heart Tumors, Histiocytosis, Langerhans Cell Hodgkin Lymphoma, Hypopharyngeal Cancer, Intraocular Melanoma, Islet Cell Tumors, Pancreatic Neuroendocrine Tumors, Kidney (Renal Cell) Cancer, Langerhans Cell Histiocytosis, Laryngeal Cancer, Leukemia, Lip and Oral Cavity Cancer, Liver Cancer, Non-Small Cell Lung Cancer, Small Cell Lung Cancer, Pleuropulmonary Blastoma, Tracheobronchial Tumor, Male Breast Cancer, Malignant Fibrous Histiocytoma of Bone, Melanoma, Intraocular (Eye), Merkel Cell Carcinoma, Mesothelioma, Metastatic Squamous Neck Cancer, Midline Tract Carcinoma, Mouth Cancer, Multiple Endocrine Neoplasia Syndromes, Multiple Myeloma/Plasma Cell Neoplasms, Mycosis Fungoides, Myelodysplastic Syndromes, Myelodysplastic/Myeloproliferative Neoplasms, Myelogenous Leukemia, Chronic (CML) Myeloid Leukemia, Nasal Cavity and Paranasal Sinus Cancer, Nasopharyngeal Cancer, Neuroblastoma, Non-Hodgkin Lymphoma, Oral Cancer, Lip and Oral Cavity Cancer, Oropharyngeal Cancer, Osteosarcoma, Undifferentiated Pleomorphic Sarcoma of Bone, Ovarian Cancer, Pancreatic Cancer, Pancreatic Neuroendocrine Tumors, Papillomatosis, Paraganglioma, Paranasal Sinus and Nasal Cavity, Parathyroid Cancer, Penile Cancer, Pharyngeal Cancer, Pheochromocytoma, Pituitary Tumor, Plasma Cell Neoplasm/Multiple Myeloma, Pleuropulmonary Blastoma, Primary Central Nervous System (CNS) Lymphoma, Primary Peritoneal Cancer, Prostate Cancer, Rectal Cancer, Retinoblastoma, Rhabdomyosarcoma, Salivary Gland Cancer, Ewing Sarcoma (Bone Cancer), Kaposi Sarcoma, Osteosarcoma, Soft Tissue Sarcoma, Uterine Sarcoma, Sezary Syndrome, Skin Cancer, Small Intestine Cancer, Squamous Cell Carcinoma of the Skin, T-Cell Lymphoma, Testicular Cancer, Throat Cancer, Oropharyngeal Cancer, Hypopharyngeal Cancer, Thymoma, Thymic Carcinoma, Thyroid Cancer, Tracheobronchial Tumor, Transitional Cell Cancer of the Renal Pelvis and Ureter, Urethral Cancer, Uterine Cancer, Endometrial Cancer, Vaginal Cancer, Vascular Tumor, Vulvar Cancer, Wilms Tumor, or any combination thereof. In some implementations, the cancer is Bladder Cancer, Breast Cancer, Colon cancer, Rectal Cancer, Endometrial Cancer, Kidney Cancer, Leukemia, Liver Cancer, Lung Cancer, Melanoma, Non-Hodgkin Lymphoma, Pancreatic Cancer, Prostate Cancer, Thyroid Cancer, or any combination thereof.

In some implementations, the subject has a tumor. In some implementations, the tumor is Atypical Teratoid/Rhabdoid Tumor, Bronchial Tumors, Carcinoid Tumor, Cardiac (Heart) Tumors, Germ Cell Tumor, Embryonal Tumor, Extracranial Germ Cell Tumor, Gastrointestinal Carcinoid Tumor, Gastrointestinal Stromal Tumors (GIST), Extragonadal Germ Cell Tumors, Ovarian Germ Cell Tumors, Heart Tumors, Islet Cell Tumors, Pancreatic Neuroendocrine Tumors, Tracheobronchial Tumor, Vascular Tumor, Wilms Tumor, or any combination thereof.

In some implementations, the peripheral blood leukocytes or sentinel lymph node leukocytes comprise a dendritic cell. In some implementations, the at least one antigen-specific T cell is a CD8 T cell or a CD4 T cell. In some implementations, the CD4 T cell is a T_{H1}, T_{H2} Or T_{H17} T cell. In some implementations, the method further comprises contacting the peripheral blood leukocytes or sentinel lymph node leukocytes of the subject with at least one effector molecule or a second delivery vehicle molecule encapsulating an mRNA encoding an effector molecule to thereby form an mRNA nanoparticle, wherein the effector molecule is a T cell reprogramming molecule, including a cytokine such as IL12, IL2, IL7, IL15, IL18, IL21, IL3, Interferons (i.e., IFNs) such as IFNα, IFNβ, or IFNγ, or Tumor Necrosis Factor a (i.e., TNF-α); a co-stimulatory molecule such as CD80, CD86, ICOS Ligand, CD70, 4-1BBL, CD40, CD40L, OX40, OX40L, TCF7, ICAM-1, LFA-1, LFA-2, LFA-3, LIGHT, or HVEM, a transcription factor, human telomerase, PU.1, CEPBA, CIITA, an HLA, β2 Microglobulin, TAP-1, TAP-2, IRF4, STAT3, or invariant chain Li; or an antibody or antigen-binding fragment thereof, including an anti-CD3 antibody (i.e., a-CD3), an a-CD28, an a-CD40, an a-OX40, an a-PD1, an a-CTLA4, an a-TIGIT, an a-LAG3, or an a-GITR; or a molecule that enhances T cell proliferation, such as IL2, IL3, IL4, IL7, IL15, IL18, 4-1BB, CD3z, CD28, an anti-PD1 antibody, or an anti-CTLA4 antibody. In some implementations, the method further comprises expanding a number of the at least one antigen-specific T cell. In some implementations, the number of the at least one antigen-specific T cell is passively expanded by exposing the T cells to the delivery vehicle molecule encapsulating the mRNA to form an mRNA nanoparticle, or an antigen-presenting cell that has been exposed to the mRNA nanoparticle, under conditions compatible with cell viability but without providing additional inputs or manipulation. In some implementations, the passively expanded T cell number is further expanded and specialized by exposure to a delivery vehicle molecule encapsulating an mRNA to form an mRNA nanoparticle comprising an mRNA encoding a T cell re-programming molecule, a co-stimulatory molecule, or a transcription factor. In some implementations, the workflow is automated and cells are periodically sampled to determine cell number, viability, specificity, phenotype, or any combination thereof, to produce a T cell therapy product. In some implementations, the control is a healthy cognate tissue, polynucleotide, polypeptide, or peptide, or is an accepted wild-type sequence of a polynucleotide, polypeptide or peptide. An accepted wild-type sequence is a wild-type sequence known in the art to be a wild-type sequence. In some implementations, the mRNA encodes a polypeptide fused to a human CD1d (i.e., hCD1d) sorting peptide, thereby increasing antigen presentation.

Another aspect of this disclosure is directed to a method comprising administering a therapeutically effective dose of antigen-specific T cells to a patient in need thereof, such as a cancer patient or patient with an infectious disease, autoimmune disease, or an inflammatory disease. In some implementations, the antigen-specific T cells are passively expanded in number in an environment compatible with cell viability without adding any additional inputs or manipulation. In some implementations, the antigen-specific T cells target tumor cells, such as cancerous cells. In some implementations, the antigen-specific T cells target cancer cells. In other implementations, the antigen-specific T cells target non-cancerous, benign tumor cells. In some implementations, the antigen-specific T cells administered to the patient are syngeneic T cells. In some implementations, the antigen-specific T cells administered to the patient are autologous T cells.

Yet another aspect of this disclosure provides a method comprising administering a therapeutically effective dose of antigen-specific T cells to an autoimmune patient in need thereof. In some implementations, the autoimmune disease is rheumatoid arthritis, Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticarial, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenia purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus erythematosus, Lyme disease, chronic Meniere's disease, Microscopic polyangiitis (MPA), Mixed connective tissue disease (MCTD), Mooren's ulcer, Mucha-Habermann disease, Multifocal Motor Neuropathy (MMN), Multiple sclerosis, Myasthenia gravis, Myositis, Narcolepsy, Neonatal Lupus erythematosus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatic, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenia purpura (TTP), Thyroid eye disease (TED), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, or Vogt-Koyanagi-Harada Disease. In some implementations, the antigen-specific T cells administered to the patient are syngeneic T cells. In some implementations, the antigen-specific T cells administered to the patient are autologous T cells.

Still another aspect of this disclosure presents a method comprising administering a therapeutically effective dose of antigen-specific T cells to a patient with an infectious disease or an inflammatory disease that is in need thereof. In some implementations, the antigen-specific T cells administered to the patient are syngeneic T cells. In some implementations, the antigen-specific T cells administered to the patient are autologous T cells.

Yet another aspect is drawn to a method comprising: contacting peripheral blood leukocytes or sentinel lymph node leukocytes from a subject exposed to an antigenic polypeptide or antigenic peptide with a delivery vehicle molecule encapsulating an mRNA to form an mRNA nanoparticle comprising an mRNA encoding the antigenic polypeptide or an antigenic fragment thereof; expanding the number of at least one antigen-specific T cell from the peripheral blood leukocytes or sentinel lymph node leukocytes; and administering an effective dose of the antigen-specific T cells to the subject, thereby boosting the immune response to the antigenic polypeptide. In some implementations, the subject is exposed to the antigen in the form of a vaccine. In some implementations of this aspect of the disclosure, the antigen is expressed in vivo from an mRNA introduced into the subject, for example by using a delivery vehicle molecule disclosed herein.

Another aspect of the disclosure is a method comprising administering a delivery vehicle molecule encapsulating an mRNA to form an mRNA nanoparticle vaccine to a subject at risk of having a disease, wherein the mRNA encodes an antigenic polypeptide or antigenic peptide derived from the subject, thereby vaccinating the subject by inducing an immune response in the subject. In some implementations, the method further comprises a second administration, or second delivery, of an mRNAnanoparticle vaccine, thereby boosting the immune response. In some implementations, the mRNA of the administered mRNA nanoparticle vaccine and the mRNA of the second delivered mRNA nanoparticle vaccine are identical mRNAs. Stated in terms of first and second delivered mRNA nanoparticle vaccines, these vaccines comprise an mRNA of the same sequence. In some implementations, the administered mRNA nanoparticle vaccine and the second delivered mRNA nanoparticle vaccine are identical mRNA nanoparticle vaccines. Again stated in terms of first and second mRNA nanoparticle vaccines, the vaccines are identical, i.e., the administered mRNA nanoparticle vaccine and the second delivered mRNA nanoparticle vaccine are comprised of identical components. Consistent with the foregoing, in some implementations of the methods of this disclosure, the administered mRNA nanoparticle vaccine comprises a first delivery vehicle molecule, and the second delivered mRNA nanoparticle vaccine comprises a second delivery vehicle molecule, and the first delivery vehicle molecule and the second delivery vehicle molecule are the same. In some implementations, the administered mRNA nanoparticle vaccine comprises a first delivery vehicle molecule, and the second delivered mRNA nanoparticle vaccine comprises a second delivery vehicle molecule, and the first delivery vehicle molecule and the second delivery vehicle molecule are different.

In some implementations of each of the methods disclosed herein, the delivery vehicle molecule encapsulates the mRNA to form a multicomponent lipitoid mRNA nanoparticle. The mRNA of multicomponent lipitoid mRNA nanoparticles encode an antigenic polypeptide or antigenic peptide, with or without encoding an effector molecule such as a T cell reprogramming molecule, a co-stimulatory molecule, a transcription factor, or an antibody or antigen-binding fragment thereof, or a molecule that enhances T cell proliferation. Delivery vehicles encapsulating mRNAs to form multicomponent lipid mRNA nanoparticles according to the disclosure comprise complexes of lipidated cationic peptide compounds in combination with further lipid components, such as structural lipids, phospholipids, and shielding lipids, which may include cationic peptide-phospholipid conjugates, known as lipitoids, or N-substituted cationic peptide compounds that are N-substituted with lipid moieties, and/or (oligo- and/or poly-)ethylene glycol moieties (referred to herein as tertiary amino lipidated and/or PEGylated cationic peptide compounds) as the cationic peptide compounds, as described in greater detail herein. These delivery vehicle molecules encapsulating mRNAs to form multicomponent lipid mRNA nanoparticles provide vehicles for mRNA delivery to cells that are highly efficient and exceed the efficiencies observed for commercially available lipid nanoparticle formulations.

One more aspect of this disclosure provides a vaccine comprising an mRNA nanoparticle comprising an mRNA encoding a peptide comprising a neo-epitope. In some implementations, the mRNA nanoparticle comprises a delivery vehicle molecule that is a multicomponent lipitoid-based nanoparticle.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### BRIEF DESCRIPTION OF THE DRAWING

FIG. 1. Flowchart of cancer vaccine epitope discovery involving selection with simultaneous generation of tumor-specific T cell product. (A) and (B) illustrate a flowchart for eliciting, inducing and expanding patient-specific, function-specific T cell product.

FIG. 2. Production of Adoptive transfer T-Cell product. (A) Flowchart for simultaneous function-specific vaccination (e.g., cancer vaccination) and peripheral blood leukocyte (PBL) harvest to generate adoptive T cell product; (B) Flowchart illustrating initial in vivo expansion and induction of function-specific (e.g., cancer-specific) T cells followed by PBL harvest for generation of adoptive T cell product.

FIG. 3. FACS sorts of activated Peripheral Blood Mononuclear Cells (PBMCs). (A) Gated on CD4+ T cell. From left: FACS plot of control treated PBMCs, Gen. 1 mRNA-activated, Gen.2 mRNA activated, peptide-activated. (B) Gated on CD8+ T cells. From left: FACS plot of control treated PBMCs, Gen. 1 mRNA-activated, Gen.2 mRNA activated, peptide-activated.

FIG. 4. Peripheral blood mononuclear cell transfection efficiencies of various peripheral blood mononuclear cell types as indicated in the figure. Transfection efficiencies were measured 24 hours after cell exposure to Thy1 mRNA nanoparticles. Histogram heights reflect the percentage of parental cells transfected.

FIG. 5. Naturally occurring antigen-presenting cells (APCs) transfected with antigen-encoding mRNA offer superior antigen presentation and T cell activation compared to conventional peptide antigen stimulation. (A) Flow cytometry of CD8 T cell activation after antigen challenge. (B) IFNγ secretion in antigen-treated or control-treated PBMC samples.

FIG. 6. (A) and (B) Antigen presentation and subsequent T cell activation were further enhanced by optimizing the antigen mRNA design by inclusion of MHC presentation enhancing sequences.

FIG. 7. T cells activated by naturally occurring APCs can kill antigen-expressing target cells and can be passively expanded to reach large numbers. (A) Robust CD8 T cell growth is evident in cultures treated with 1 μg mRNA encoding pp65 with MHC presentation-enhancing sequences. Both viability and cell numbers were superior to cells treated with peptide. (B) Activated and expanded CD8 T cells recognized and killed T2 target cells only when T2 target cells were pulsed with CMV-pp65 antigens. (C) Significantly better killing efficacy is seen in CD8 T cells isolated from cultures that were treated with mRNA compared to peptides.

FIG. 8. Naturally occurring antigen-presenting cells (APCs) transfected with antigen-encoding mRNA offer superior antigen presentation and T cell activation compared to conventional peptide antigen stimulation in PBMCs from cervical dysplasia patients. (A) Flow cytometry of CD8 T cell activation after antigen challenge. (B) Robust CD8 T cell growth is evident in cultures treated with 1 μg mRNA encoding HPV16 antigen mRNA with MHC presentation-enhancing sequences. Both viability and cell numbers were superior to cells treated with peptide.

### DETAILED DESCRIPTION

The disclosure provides immunotherapeutic methods that combine disease antigenic peptide (e.g., cancer antigenic peptide, such as a tumor antigenic peptide) identification with the robustness and flexibility of mRNA nanoparticles. In one example, the mRNA nanoparticle comprises a delivery vehicle molecule encapsulating at least one mRNA encoding at least one antigenic peptide. The term “encapsulating” herein connotes any suitable degree of coverage of the delivery vehicle molecule around the mRNA(s). For example, the encapsulation may refer to the mRNA completely covered and surrounded by the delivery vehicle molecule, or partially covered and surrounded by the delivery vehicle molecule (e.g., substantially covered).

The mRNA nanoparticle formed at least in part by encapsulating the mRNA with at least one delivery vehicle molecule may be sued to transport the encapsulated mRNA, which encodes at least one antigenic peptide to antigen presenting cells, such as dendritic cells, for expression, processing, and presentation to T cells of the subject ex vivo. The methods facilitate the development of optimized mRNA encoding a disease antigenic peptide, such as a tumor antigenic peptide. A therapeutic or prophylactic mRNA of this disclosure, which encodes at least an antigenic polypeptide or antigenic peptide, may be encapsulated within a delivery vehicle molecule to generate an mRNA nanoparticle. mRNA nanoparticles according to this disclosure may also provide effector molecules, which may be encoded by one or more mRNAs encapsulated in the delivery vehicles to form mRNA nanoparticles, for reprogramming and expansion of tumor-infiltrating T lymphocytes. Further, the disclosure provides combinations of effector and tumor-antigen mRNAs to overcome suppression, enhance activation, ensure expansion, and/or induce T cell reprogramming. The materials and methods disclosed herein provide for effective and efficient identification and expansion of antigenic peptide-specific T cells targeting disease, such as cancer antigenic peptide-specific (e.g., tumor antigenic peptide-specific) T cells for adoptive cell therapy (i.e., ACT).

The immunotherapeutic methods of this disclosure harness the power of a subject's own immune response to treat diseases such as cancer, autoimmune diseases and inflammation. The methods focus on self-antigens in the form of antigenic polypeptides or peptides, and deliver delivery vehicle molecules encapsulating mRNAs to form mRNA nanoparticles, wherein the mRNAs encode such polypeptides and peptides to allow cellular physiology to process and express those encoded products in a manner that yields surprisingly effective immune responses. This disclosure further provides methods for boosting that immune response by delivering to a subject with a disease, such as cancer, an autoimmune disease or inflammation, a delivery vehicle molecule encapsulating an mRNA to form an mRNA nanoparticle, wherein the mRNA encodes an antigenic polypeptide or peptide. In some implementations, the encapsulated mRNA used to boost the immune response is the same (i.e., has the same sequence) as the encapsulated mRNA used to elicit an initial immune response. The delivery vehicle molecule encapsulating the mRNA in forming the mRNA nanoparticle that is used to boost an immune response may be the same (i.e., have identical components) or different from the delivery vehicle encapsulating the mRNA to form the mRNA nanoparticle used to elicit the initial immune response.

In addition, the materials and methods disclosed herein provide for the identification of functional T cell receptor (i.e., TCR) sequences for generation of transgenic T cell therapy products. Effector molecules suitable for use in the methods of this disclosure include, but are not limited to, the example effector molecules identified in Table 1.

TABLE 1

| | Co-stimulatory | Transcription Factors | |
|---|---|---|---|
| Cytokines | Receptors | and other proteins | Antibodies |
| IL12 | CD80 | Human telomerase | a-CD3 |
| IL2 | CD86 | PU.1 | a-CD28 |
| IL7 | ICOS Ligand | CEPBA | a-CD40 |
| IL15 | CD70 | CIITA | a-OX40 |
| IL18 | 4-1BBL | HLAs | a-PD1 |
| IL21 | CD40 | β2 Microglobulin | a-CTLA4 |
| Type I IFNs | CD40L | TAP-1/2 | a-TIGIT |
| TNF-α | OX40 | IRF4 | a-LAG3 |
| IFNγ | OX40L | STAT3 | a-GITR |
| IL3 | TCF7 | Invariant chain Li | |
| | ICAM-1 | | |
| | LFA-1,2,3 | | |
| | LIGHT | | |
| | HVEM | | |

Antigenic peptides (e.g., tumor antigenic peptides) contemplated for use in the methods of this disclosure include, but are not limited to, antigenic peptides modified to include protease-cleavable linkers between independent antigen coding sequences, the inclusion of multiple antigenic peptides (same or different antigenic peptides) in the same mRNA molecule, stabilized cytokines, stabilized receptor molecules, enhanced secreted growth factors, constitutively active signaling domains, the inclusion of Major Histocompatibility Complex I (i.e., MHC I) and/or MHC II sorting sequences such as MITD and hCD1d, endoplasmic reticulum retention sequences such KDEL, secretion signals/leader peptides, T-helper epitopes such as PADRE, cell-specific untranslated sequences, and/or cell-specific codon optimization. Any of these molecules can undergo a suitable optimization process before use. Any or all of these optimizations may be applied individually or in any combination to optimize an antigenic peptide for use in the methods of the disclosure.

It is contemplated that subject-specific (e.g., patient-specific) biomaterials may be obtained from any organ, tissue or cell source of the subject. Example sources for such biomaterials are the circulatory system, e.g., whole or fractionated blood, the lymphatic system (e.g., lymph nodes such as sentinel lymph nodes), and disease tissue (e.g., tumor tissue).

The use of self-mRNA-encoded antigenic peptides to stimulate or activate an immune response to disease in applications of personalized medicine provide the benefit of avoiding the onerous requirement for ex vivo generation of antigen-presenting cell (e.g., APC) populations, the ability to reduce, or in some instances even eliminate, the need for exogenously introduced growth factors and cytokines, the ability to avoid peptide synthesis and conjugation, the implementation of methods not plagued by the suboptimal presentation of antigenic peptides, the suboptimal co-stimulation of T cells, and/or the suboptimal generation of effector cell subtypes, the reduction, or in some instances even elimination, of the problem of immunosuppression, and/or T cell exhaustion that, in some instances, afflicts other forms of immunotherapy.

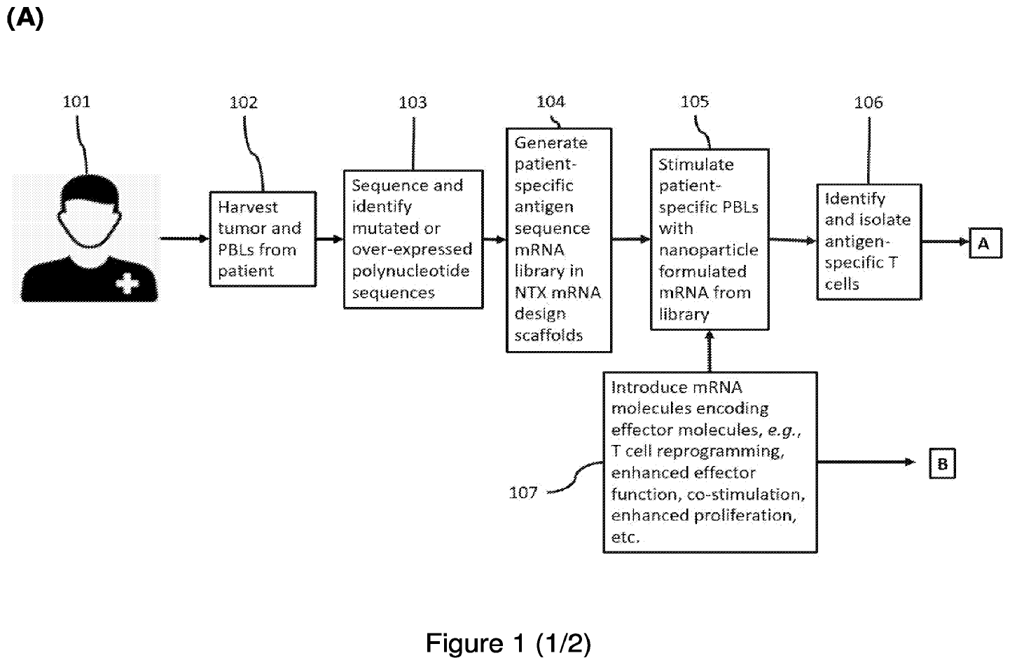

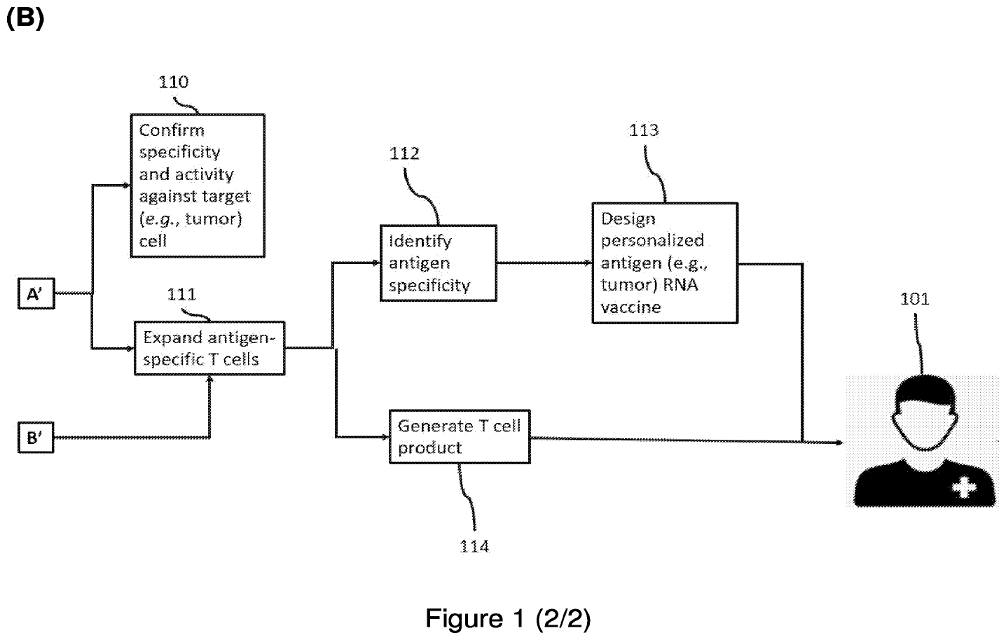

Turning now to FIG. 1(A) as an illustration of an example workflow involved in the methods of this disclosure, subject 101 (e.g., a human patient) is subjected to harvest step 102, which provides a biological sample from which peripheral blood leukocytes, and tumor cells if the subject is to be treated for a tumor, are harvested. The partial or complete genome of harvested cells is then subjected to a sequence and identification step 103 to identify mutated or over-expressed polynucleotide sequences. It is noted that the subject may be any mammal, and human is only one example. It is noted that in some instances, the sample may be provided pre-harvested, and thus the workflow may start at step 103. The identification of mutated and/or over-expressed sequences is followed by a subject-specific (e.g., patient-specific) mRNA library construction step 104, wherein the library is generated using NTX mRNA design scaffolds. The mRNA library is then used in a stimulation step 105 to stimulate subject-specific (e.g., patient-specific) PBLs using delivery vehicles encapsulating mRNAs to form mRNA nanoparticles formulated as described herein, wherein during the process of generating delivery vehicles encapsulating mRNAs, the formed mRNA nanoparticles may also encapsulate mRNAs encoding effector molecules in a delivery vehicle loading step 107, which may also be viewed as an mRNA nanoparticle loading step 107. Following stimulation of subject-specific (e.g., patient-specific) PBLs with delivery vehicles encapsulating mRNAs to form mRNA nanoparticles, an identification step 106 is performed in which specific T cells are identified and partially or completely isolated.

The workflow depicted in FIG. 1(A) continues as shown in FIG. 1(B), where the identified and isolated T cells are subjected to a confirmation step 110 wherein the specificity and activity against a target cell (e.g., a tumor cell) are confirmed. The collection of identified and isolated T cells, which may also include PBLs exposed to delivery vehicle molecules encapsulating mRNAs to form mRNA nanoparticles, wherein the mRNAs also encode effector molecules, undergo an expansion step 111 in which antigen-specific T cells are expanded. Expanded T cell populations are then subjected to an antigen identification step 112 to identify the antigen specificity of the T cells. T cells for which antigen specificity has been determined are subjected to a personalized antigen design step 113 to tailor an RNA (e.g., mRNA) vaccine to a particular subject (e.g., patient). The population of expanded T cells is also subjected to a T cell generation step 114 to produce T cell product, which is then available for delivery to subject 101 (e.g., patient 101). It is noted that the steps described in FIG. 1(A)-1(B) may be performed by the same person or different persons.

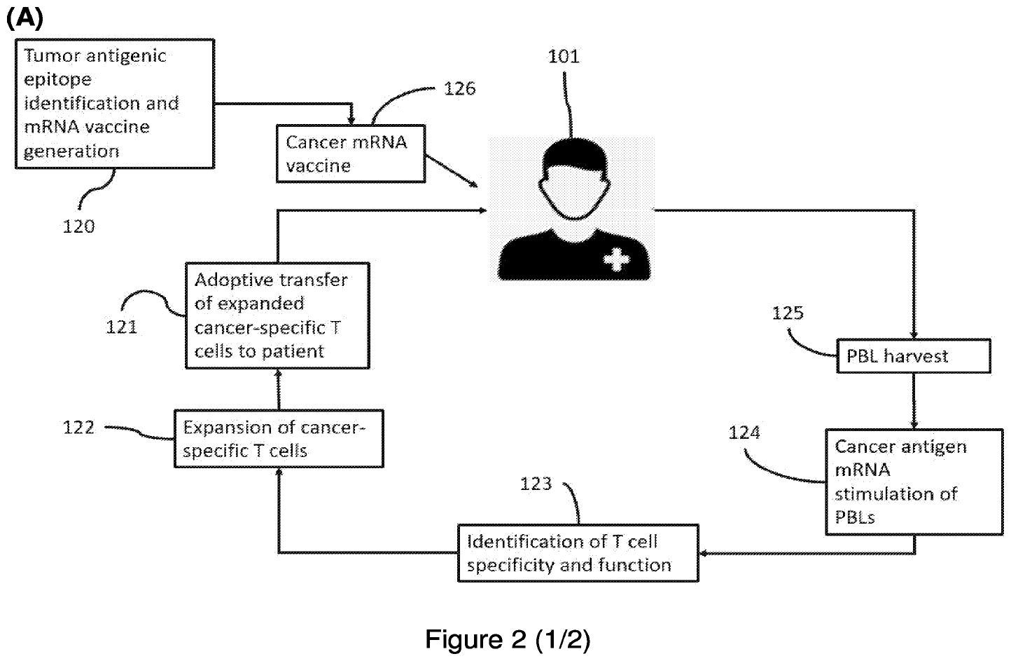

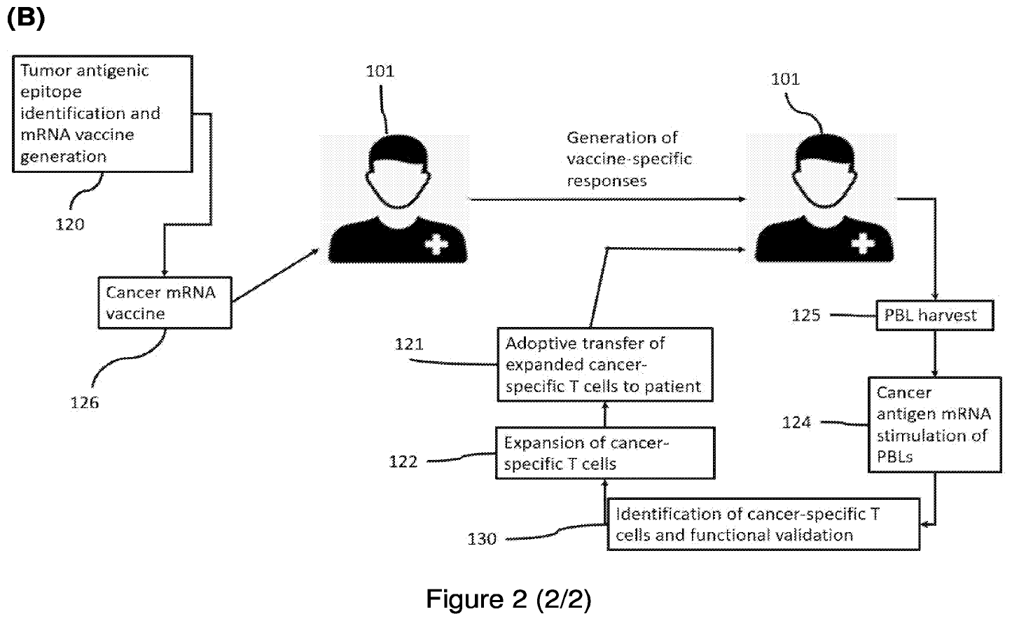

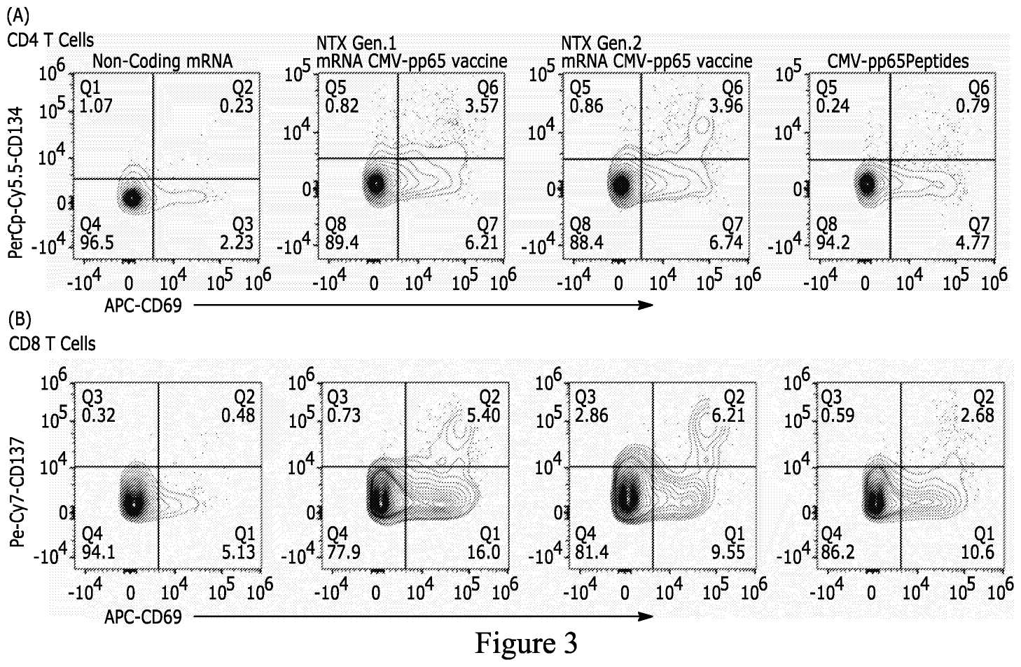

FIG. 2(A) illustrates an example workflow for the simultaneous function-specific vaccination (e.g., cancer vaccination) and peripheral blood leukocyte (PBL) harvest to generate adoptive T cell product. An epitope identification and vaccine generation step 120 is performed in which tumor antigenic epitopes are identified and an mRNA vaccine is generated based on the identifications. The mRNA vaccine is then subjected to administration step 126 in which, e.g., a cancer mRNA vaccine is administered to a subject. The subject in this disclosure may refer to any patient in need of the treatment described herein. At the same time, a PBL harvest step 125 is performed on the subject and the harvested PBLs undergo a stimulation step 124 in which mRNAs encoding known cancer antigens are used to stimulate the subject's PBLs. The stimulated PBLs are then analyzed in a T cell identification step 123 to identify T cell specificity and function. Identified T cells of interest are then subjected to an expansion step 122 to yield cancer-specific T cells. The expanded cancer-specific T cells are then used in an adoptive transfer step 121 to provide the subject with cancer-specific T cells tailored to address the subject's specific cancer.

FIG. 2(B) provides an alternative example flowchart to the one illustrated in FIG. 2(A). FIG. 2(B) show the workflow involved in using discrete protocols to achieve the in vivo expansion and induction of function-specific (e.g., cancer-specific) T cells followed by PBL harvest for generation of adoptive T cell product. Using this approach, an epitope identification and vaccine generation step 120 is initially performed to yield a cancer mRNA vaccine that is administered to subject 101 (e.g., patient 101). Subject 101 then generates vaccine-specific responses, and PBLs from the vaccinated subject 101 are obtained in PBL harvest step 125. Harvested PBLs undergo a stimulation step 124 wherein PBLs are stimulated by mRNA-encoded cancer antigens. Next an identification and validation step 130 is performed to identify and characterize target-specific (e.g., cancer-specific) T cells. The target-specific T cells are then subjected to an expansion step 122 to increase the number of such cells, and the expanded target-specific T cells are then used in an adoptive transfer step 121 by administering the expanded target-specific (e.g., cancer-specific) T cells to the subject. It is noted that any, including all, parts of the workflow described herein may be automated. For example, the cells may be periodically sampled to determine cell number, viability, specificity, phenotype, or any combination thereof, to produce a T cell therapy product. The automation may involve for example at least one processor and the necessary hardware and algorithms.

The following disclosure further explains these steps may be implemented in a variety of contexts.

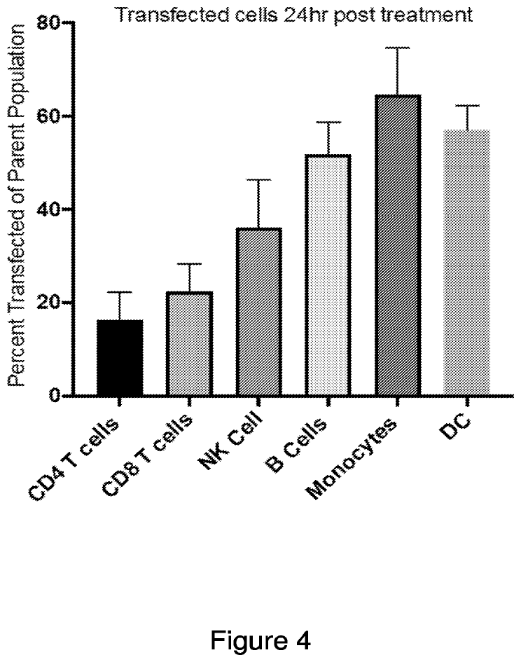

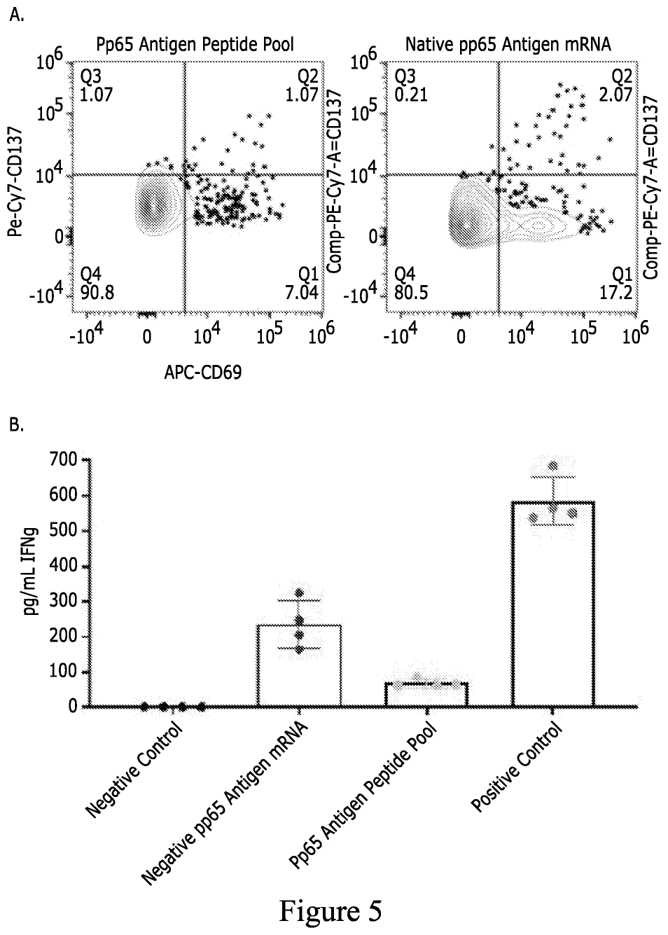

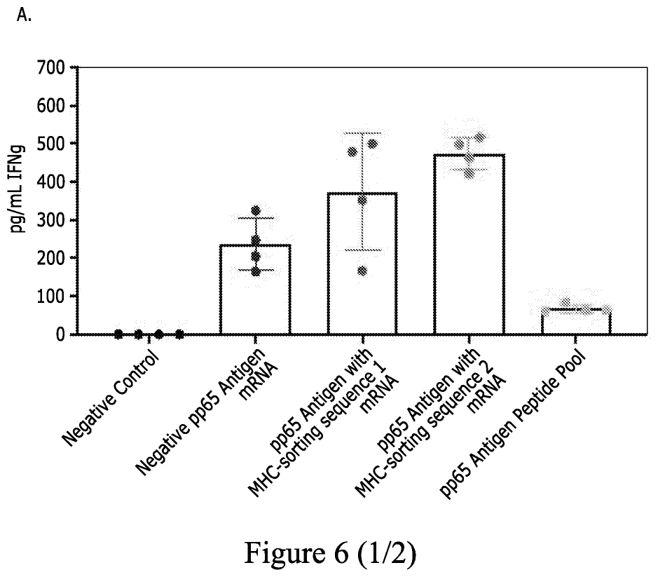

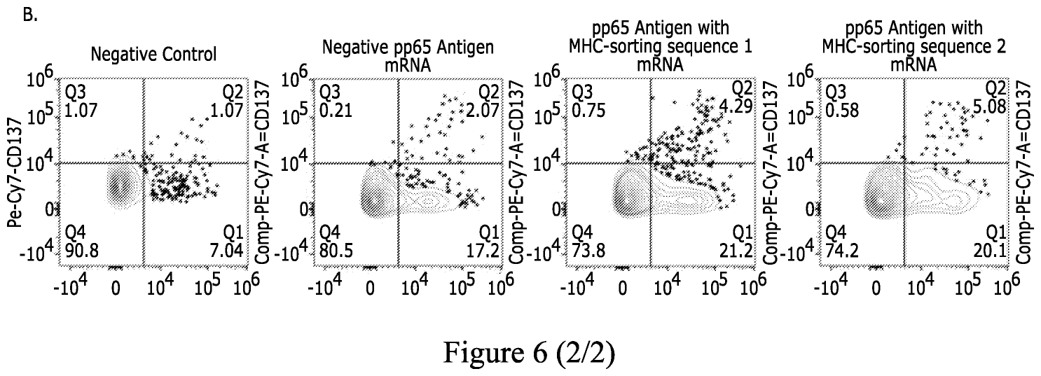

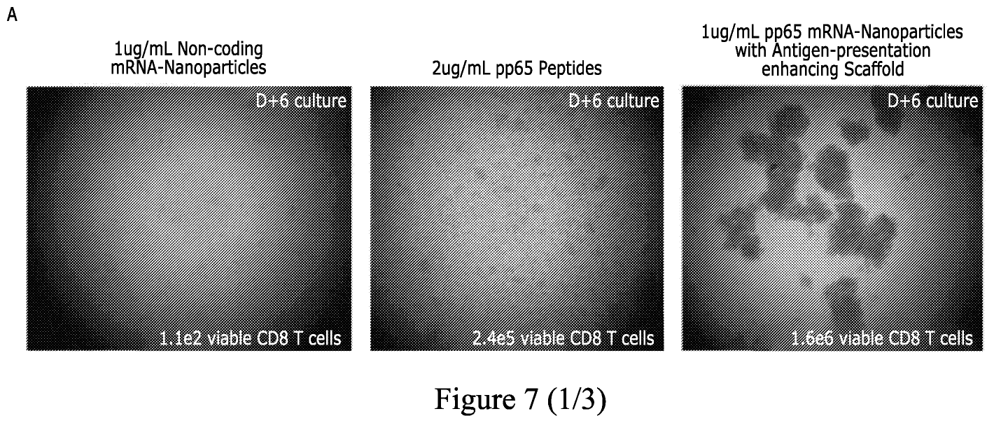

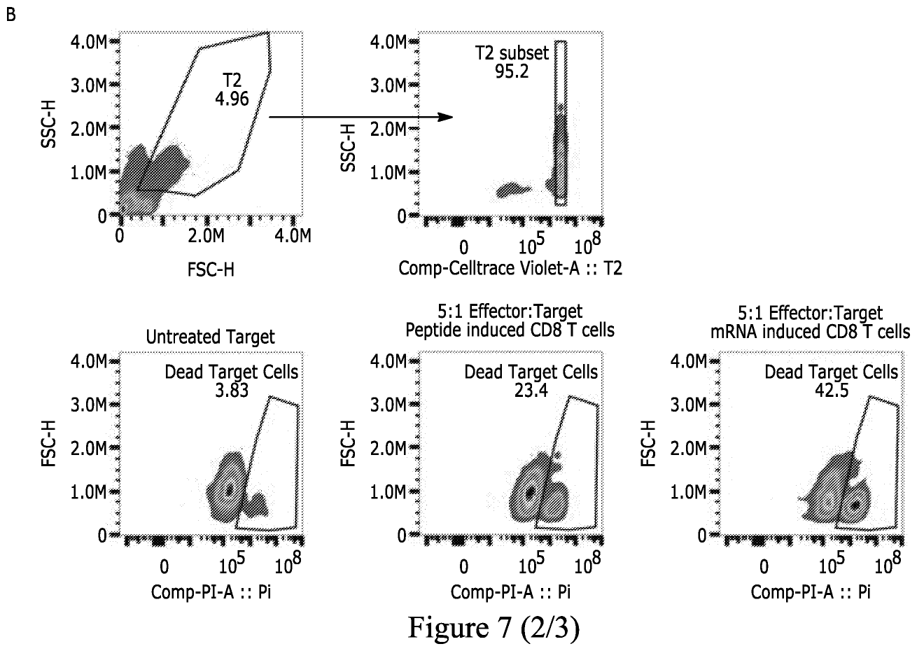

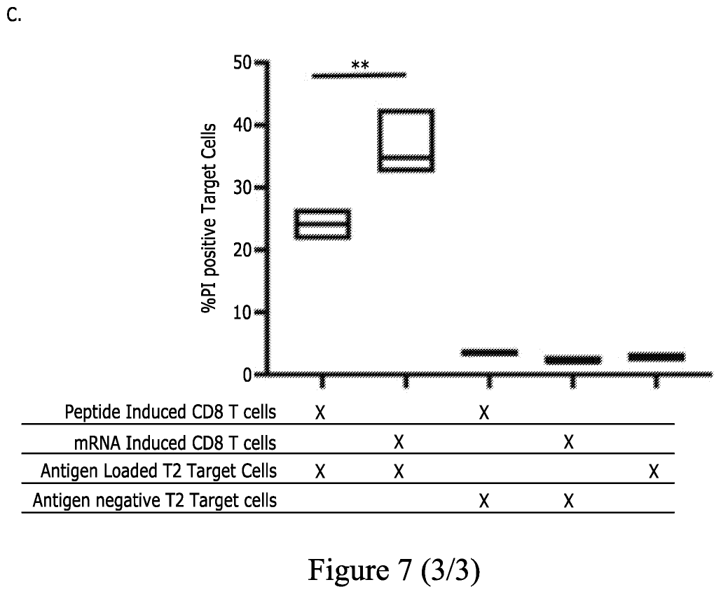

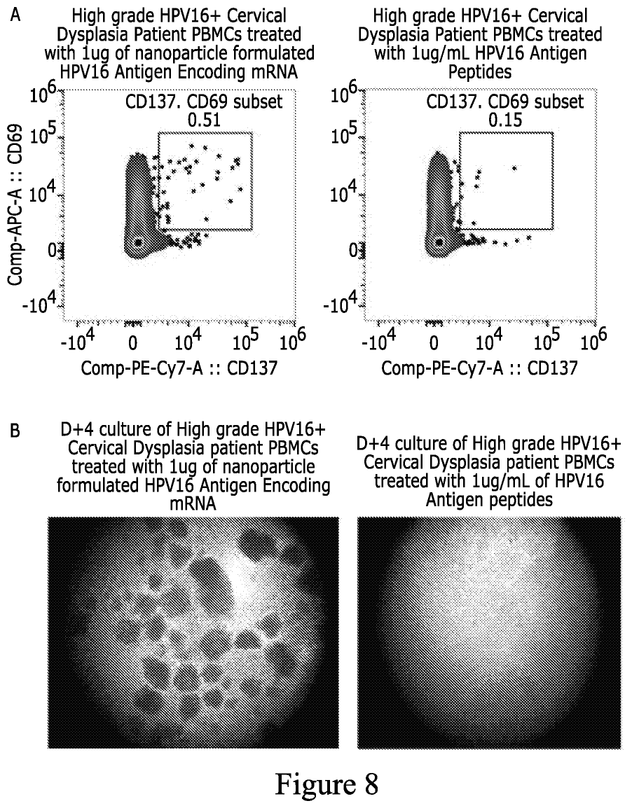

Disclosed herein are experimental results establishing that delivery vehicle molecules (in some instances referred to as “delivery vehicles” for short) effectively deliver mRNA cargo in the form of an mRNA nanoparticle to living cells in a mixed population of primary immune cells in vitro (FIG. 4). The data also show the surprising result that naturally occurring APCs transfected with mRNA encoding an antigenic peptide offer superior antigen presentation and T cell activation compared to conventional peptide antigen stimulation (FIG. 5). The mRNA-based presentation of antigenic peptides allows the cells of the subject to process expression products to yield the antigenic peptide(s) that elicit the personalized immune response. It is also noted that the methods according to the disclosure provide for antigen presentation and subsequent T cell activation can be further enhanced by optimizing the antigenic peptide-encoding mRNA design by inclusion of MHC sorting sequences (FIG. 6). These methods take advantage of T cells activated by naturally occurring APCs that have processed and translated mRNA and processed and presented the expressed antigenic peptides, enabling the T cells to be passively expanded to reach large numbers (FIG. 7A). Moreover, T cells activated by naturally occurring APCs that have processed and translated mRNA, leading to the expression, processing and presentation of encoded antigenic peptides, can kill a target cell expressing the same antigen in a MHC-restricted manner (FIG. 7B). Further, antigenic peptides encoded by mRNA result in a more polyclonal, and therefore more robust, T cell response compared to the exogenous administration of peptides as antigenic material. As shown in FIGS. 8(A)-8(B), naturally occurring antigen-presenting cells (APCs) transfected with antigen-encoding mRNA offered superior antigen presentation and T cell activation, compared to conventional peptide antigen stimulation in PBMCs from cervical dysplasia patients. FIG. 8(A) shows flow cytometry result of CD8 T cell activation after antigen challenge, and FIG. 8(B) shows robust CD8 T cell growth evident in cultures treated with 1 μg mRNA encoding HPV16 antigen mRNA with MHC presentation-enhancing sequences. The results showed that both viability and cell numbers were superior to cells treated with peptide.

Placing the materials and methods of this disclosure in context, it is noted that generating a productive T cell product for adoptive T cell therapy involves paying attention to aspects such as levels of antigen expression, specificity to cancer tissue, and whether efficient presentation of the antigen is possible in association with a specific human leukocyte antigen (HLA)-molecule. These considerations may complicate target selection and confer risks such as insufficient targeting efficacy or off-target effects. The in vitro production of the T cell product may add another layer of difficulties. Factors such as the choice of cytokines during T cell culture may be important, as different T cell phenotypes are known to have different potencies in vivo. Additional considerations involved in developing a T cell therapy may include the selection of a source of T cells, the selection of a source of antigen-presenting cells, designing an approach to mitigate or otherwise address the phenomenon of immunosuppression, the approach taken to select appropriate antigens, the protocol to adopt in expanding responsive T cells, the methods to secure sufficient co-stimulation of T cells to ensure a therapeutically useful activation of T cells, the selection of effector phenotype, and the real threat of T cell exhaustion.

### Antigen Selection

The choice of target antigenic peptide(s) for T cell adoptive cell therapy (i.e., ACT) may be important to the reliable achievement of treatment success. The methods of this disclosure are flexible in being suitable for use with any of at least three main target categories, i.e., (1) therapies with an unknown target, (2) therapies that target known tumor-associated antigen (TAAs), and (3) therapies that target neoantigens specific to one individual tumor.

### Unknown Antigens

Methods of this disclosure that target unknown antigens are commonly based on the sourcing of T cell populations enriched for antitumor specificity. One of the well-known and extensively studied is the tumor infiltration lymphocyte, or TIL, methodology, where isolation of T cells infiltrating the tumor tissue is performed. The basis for harvesting this specific T cell population is the enrichment of tumor-specific T cells in the tumor tissue. The culture process used in many published studies follows the rapid expansion protocol (REP), which includes a first step, sometimes called the pre-REP, where TILs are isolated and expanded from digested tumor tissue and cultured in interleukin 2 (IL-2) to obtain a starting batch of TILs. Subsequently, in the REP culture step, the TILs are re-stimulated via TCR stimulation (monoclonal anti-CD3) together with irradiated allogeneic feeder cells.

In terms of treatment, subjects such as patients are often lymphodepleted following removal of the lymphocytes used in preparation of the antigen-presenting cells, and ultimately the therapeutic T cell products, described herein. In some cases, the lymphodepleted patients receive radiotherapy before T cell product infusion. T cell infusion is often accompanied with an in vivo administration of IL-2.

The treatment protocols of this disclosure also include lymphodepletion with chemotherapy and, in some cases, radiotherapy before T cell infusion and in vivo IL-2 administration after infusion. A variety of dosage levels of IL-2 are contemplated, but relatively low dosages minimize undesired side effects. In one example, lymphodepletion is performed to improve the survival and localization of infused T cells, such as TILs, through ensured access to homeostatic cytokines for the infused TILs, reduction of Tregs, and stimulatory effects on antigen-presenting cells (APCs).

Another approach targeting unknown antigens is the Sentoclone® method, where tumor-draining sentinel lymph nodes provide the source of autologous T cells. Sentinel nodes, like tumor tissue, have T cell populations enriched for tumor-specific T cells. The isolated T cells are stimulated with autologous tumor homogenate, expanded in vitro and re-infused without previous lymphodepletion or adjuvant IL-2.

### Tumor-Associated Antigens

The methods of this disclosure are also suitable for use with delivery vehicle molecules encapsulating mRNAs to form mRNA nanoparticles, wherein the mRNAs encode tumor-associated antigens. Identification and targeting of tumor-associated antigens (TAAs) is another strategy for cancer immunotherapy. In one example, it is desirable for TAAs to be homogeneous, highly stable, and specifically expressed by tumors cells (i.e., not found in healthy tissues), present in many patients, recognized by T cells, and subsequently able to elicit T cell cytotoxicity. Naturally occurring TAA-specific T cells can be sourced from a patient's blood and expanded using known tumor-associated peptide antigens (e.g., PRAME, MAGEA4, SSX2, Survivin, and NY-ESO-1), in combination with antigen presenting cells (APCs) and cytokines, in various implementations of the disclosed methods.

### Neo-Antigens

Newly formed tumor-specific antigens, also called neoantigens herein, arise from non-synonymous mutations and other abnormal genetic modifications in a subject's body. One benefit of targeting neoantigens over TAAs is that they are highly specific for the cancer cells of one individual tumor. They are not found in normal tissues, as the mutations do not occur in germline DNA. They can thus be differentiated from normal self-antigens and recognized as non-self by T cells. They are specific to a single individual tumor, compatible with a personalized approach to treatment.

The characteristics of the mutations influence therapy outcome, as vaccination with a neoantigen based on a single mutation has been found sufficient to elicit positive clinical responses. Also, cancers with a high neoantigen load, such as cutaneous malignant melanoma or non-small-cell lung cancer, generally respond well to checkpoint inhibitor therapy, which is also contemplated in combination with the administration of therapeutic T cell products, such as autologous T cells targeting cancer (e.g., tumor) cells.

Several methods have been developed to facilitate prediction of which neoantigens have the potential to elicit a positive immune response, and these methods are contemplated for use in combination with the development of therapeutic T cell products targeting neoantigens, such as cancer neoantigens. Mutations discovered using major databases such as COSMIC or The Cancer Genome Atlas, many of which are cancer-driver mutations, are contemplated for use as neoantigens in methods of the disclosure. The advantage of focusing on driver mutations for neoantigen-based immunotherapy is unclear, however. Recent computational analyses of the mutational landscape for binding to both HLA class I and HLA class Il molecules indicate that driver mutations carry an overall lower affinity than random mutations. Next-generation sequencing (NGS) is also contemplated as a tool for identifying neoantigens through the systematic and personalized prediction of neoantigens for individual patients. NGS for neoantigen prediction may be based on massive parallel sequencing of either all known DNA sequences in the genome coding for proteins (Whole Exome Sequencing, WES) or presently available RNA Transcripts (Transcriptome sequencing, RNA-seq) of a chosen tissue. Efforts to use NGS to identify neoantigens is aided by available neoantigen prediction pipelines, including pVAC-Seq, MuPeXI, TIMiner, and OpenVax. Most neoantigen prediction pipelines rely on WES for normal and tumor DNA combined with RNA-seq for tumor RNA, where WES traditionally is used to identify mutations only found in tumor DNA, and RNA-seq is used to verify the expression of the corresponding mRNA transcript. In addition to expression, RNA-seq also reveals additional information that is not visualized by WES, such as alternate splicing variants or transcriptional errors.

The next step in neoantigen identification may include predicting the binding of putative epitope candidates to the corresponding HLA molecules from the individual patient. The NetMHC series (version 4.0) aids in prediction of peptide binding across class I HLA alleles. Several studies have indicated, however, that the actual number of predicted peptides to be presented on HLA molecules might be lower than 5%. Antigen presentation on HLA class Il complexes is contemplated for the methods disclosed herein that are focused on neoantigen-based T cell therapies of diseases such as cancer. Neoantigens presented on HLA class Il complexes promote recognition of tumor epitopes and antitumor activity as well as presentation on HLA class I complexes. Thus, class Il molecules as well as class I molecules are included in predicting neoantigens for use in the methods of the disclosure. Yet another approach is to look at proteasome-induced splice variants of peptides. These neoantigens do not align with the original peptide chain because the splicing happens post-translation.

To overcome the hurdles associated with conventional prediction pipelines, machine-learning algorithms that correlate results concerning neoantigen peptide fitness and clinical response from several datasets simultaneously may be used to improve neoantigen prediction. The Neopepsee machine-learning platform may be used to improve sensitivity and specificity.

Neoantigens can be used in T cell ACT strategies either to stimulate and/or select autologous tumor-specific clones or as a template for the production of a tumor-specific TCR. The neoantigen concept is contemplated as applicable to a sentinel node-based T cell ACT. In such implementations, neoantigen peptides are coupled to paramagnetic beads that are added to a lymph node cell culture, and the APCs from the lymph node process the neoantigens and present them to the T cells, causing specific expansion. Neoantigen-reactive T cell clones are identified and their TCRs are sequenced and used in TCR-transgenic therapies according to the disclosure. In some implementations of the methods of the disclosure, neoantigens identified from patient tumor tissue are used to screen healthy donor T cells for reactive clones because autologous TILs are often few in number and functionally suppressed. The identified neoantigen-specific TCRs are then used for transgenic transfer into the desired T cell population. Other implementations of the methods of this disclosure involve TIL therapies, where TILs expressing PD-1 and/or activation markers OX40 and 4-1BB (and thus likely to be tumor-reactive) are sorted by flow cytometry, cultured, and co-cultured with APCs pulsed with neoantigen peptides. Responding clones are analyzed by TCR sequencing to obtain a TCR template, analogous to the method described above.

In vivo induction of T cell responses is highly dependent on interactions with professional antigen-presenting cells (APCs), in particular dendritic cells (DCs), which present tumor-specific antigens. The methods according to the disclosure are well-suited for such in vivo applications because the delivery vehicle molecules encapsulating mRNAs encoding antigenic peptides to form mRNA nanoparticles are administered to a subject in need of treatment without bias. Thus, professional APCs are not disadvantaged in taking up the delivery vehicle molecules encapsulating mRNAs in forming mRNA nanoparticles that yield antigenic peptides in proper presentation form to stimulate an immune response. In fact, natural APCs, in particular DCs, are well-equipped to induce efficient activation and expansion of tumor antigen-specific naïve T cells, which can lead to induction of large populations of T cells, including CD8⁺ cytotoxic T lymphocytes (CTLs) that can kill cancer cells presenting a specific antigen. In some examples, the use of natural APCs in cancer treatment is associated with a beneficial clinical outcome with minor adverse side effects, emphasizing the promise of active immunotherapy.

Conventional use of natural APCs, such as DCs, has uncovered several limitations. Lack of knowledge of the optimal antigen-loaded DC combined with deleterious effects of immunosuppressive factors in the tumor microenvironment may be responsible for the mixed results observed in clinical trials. In addition, in some instances, isolation and ex vivo stimulation of autologous DCs may be time-consuming and expensive, and the quality of ex vivo-generated DCs can be variable. The methods of this disclosure rely on delivery of delivery vehicle molecules encapsulating mRNAs to form mRNA nanoparticles, wherein the mRNAs encode antigenic peptides that allow a subject's own naturally occurring APCs to process the mRNA and express protein to yield a presentation of antigenic peptide that activates that subject's own T cells, thereby treating the disease such as cancer. mRNA structure

Antigenic peptides of this disclosure are encoded in mRNAs that, at minimum, include a coding region for the portion of a peptide participating in the structure of an antigen along with sufficient expression control sequences to allow the mRNA to be expressed in at least on cell type, such as a leukocyte capable of presenting an antigenic peptide for recognition by a subject's immune system. Thus, an mRNA of this disclosure may have a coding region for an antigenic peptide, a ribosome binding site (e.g., a Kozak sequence), and a stop codon, at a minimum. The mRNA of this disclosure may also contain at least one untranslated region (i.e., UTR), such as a 5′ UTR or a 3′ UTR. In several implementations, the mRNA has a 5′ UTR and a 3′ UTR. Additionally, mRNAs of this disclosure may encode effector molecules for reprogramming and expansion of T cells, such as tumor-infiltrating T lymphocytes (i.e., TILs). Also, mRNAs of this disclosure may additionally encode an MHC presentation-enhancing sequence. More broadly, the one or more of the effector coding regions described herein may be included in the mRNAs of the disclosure. Also, as noted elsewhere herein, mRNA nanoparticles of this disclosure may contain more than one type of mRNA, such as an mRNA encoding one or more antigenic peptides and another mRNA encoding one or more effector molecules, alone or in combination with at least one coding region for an antigenic peptide. In some examples, the mRNAs of this disclosure may be larger than the minimal size to encode an expressible antigenic peptide, with the additional RNA sequence(s) facilitating the intracellular processing of the mRNA by leukocytes of the subject in the process of expressing and presenting the antigenic peptide.

mRNA Nanoparticles

The delivery vehicle molecules disclosed herein encapsulate mRNAs to form mRNA nanoparticles. The nanoparticle component of the mRNA nanoparticles of the disclosure are small particles of matter generally having dimensions between 1-300 nanometers, such as a range of 60-150 nanometers, which is smaller than the typical size of a microparticle having dimensions of 1-1,000 um. The mRNA nanoparticles can either be organic (e.g., a liposome), inorganic (e.g., gold, silver, or platinum), or hollow. In general, mRNA nanoparticles contemplated include any compound or substance with a capacity to deliver an mRNA to the cells of a subject, either ex vivo (e.g., in vitro) or in vivo. Suitable compounds or compositions found in an mRNA nanoparticle of this disclosure include, without limitation, aliposomal particle, a polymer-based particle (e.g., a poly (lactic-co-glycolic acid) (PLGA) particle), insulator particle compositions, and a dendrimer (organic versus inorganic), any of which may comprise an mRNA within, in accordance with the disclosure.

### Vaccine Cassettes

A vaccine is a substance used to stimulate the production of antibodies and provide immunity against one or several diseases, prepared from the causative agent of a disease, its products, or a synthetic substitute. Disclosed herein are novel constructs and scaffolds for the generation of vaccine compositions. These vaccine compositions comprise a scaffold which carries or conveys an antigenic payload. The combination of the vaccine scaffold and antigenic payload is termed a vaccine cassette. As used herein a “vaccine cassette” is a polynucleotide (or its encoded polypeptide) encoding a vaccine scaffold and an antigenic payload which collectively function as a vaccine. Vaccine cassettes may be configured for administration directly or to be encoded in one or more polynucleotides for expression in a cell and may be encoded in DNA, RNA or mRNA for administration.

### Vaccine Scaffolds

Vaccine scaffolds of the present disclosure may be derived from one or more regions of one or more parental polypeptides, e.g., receptor molecule(s). Such parental molecules may include, but are not limited to, CD1, LDLR, LDLRP and/or LRP1 families of receptors or proteins. The disclosure contemplates implementations in which the mRNA cargo is encapsulated by delivery vehicle molecule to form an mRNA nanoparticle. The delivery vehicle molecule may comprise peptoid-based lipid materials to provide versatile and effective delivery vehicles for mRNA cargoes; this is described further below.

In some implementations, the parental molecule is selected from the CD1 glycoprotein family of receptors. CD1 proteins are encoded in a locus on human chromosome 1. This region encodes five CD1 isoforms (CD1a-e). These proteins are expressed at the cell surface and function as antigen-presenting molecules, except for CD1e, which is only expressed intracellularly and is involved in processing and editing lipid for presentations by the other human CD1 isoforms. The CD1 isomers traffic around the cell by association with a variety of chaperones, such as calnexin, calreticulin and even B2M. The newly synthesized unoccupied CD1 isomers egress to the plasma membrane from the ER and Golgi, followed by internalization and entry into different compartments through tyrosine-based sorting motifs that permit their binding with adapter protein complex 2 and 3, which facilitates entry into a variety of endosomal compartments (early endosomes, recycling endosomes, late endosomes) and lysosomes, ultimately undertaking a similar trafficking pathway to that of MHC I molecules. Furthermore, CD1 isomers traffic via these endosomal compartments to load antigen and, in many instances, CD1 and MHC I and MHC II molecules are detected within the same compartment.

According to this disclosure, vaccine scaffolds comprise the following formula:

5′UTR--[[Signal/Leader]-[(An1)n-Xo-(An2)p]-[TMD]-[CYD]]-3′ UTR-PolyA

where “UTRs” are the untranslated regions located at the 5′ and 3′ ends of a standard mRNA construct and “PolyA” refers to the polyadenylation site of the mRNA;
[Signal/Leader] refers to any signal or leader or sorting sequence in frame with, and upstream of, the antigenic payload region;
[(An1)n-Xo-(An2)p] refers to any antigenic payload region comprising a first antigenic payload (An1) which may be duplicated “n” number of times, a spacer or linker region (X) which may be optionally absent or repeated “o” number of times, and optionally a second antigenic payload (An2) which, when present, may be repeated “p” number of times; TMD refers to all or a portion of a transmembrane region from one or more CD1, LDLR, LDLRP and/or LRP1 proteins; and
CYD refers to all or portion of a cytoplasmic region from one or more CD1, LDLR, LDLRP and/or LRP1 proteins.

In some implementations, the vaccine scaffolds of the disclosure include one or more of the signal sequence and/or cytoplasmic sorting signal of CD1, LDLR, LDLRP and/or LRP1 isomers to facilitate antigen routing into the endosomal and/or lysosomal compartments, ultimately allowing the processing and loading of MHC Class I and MHC Class Il molecules.

In some implementations, the signal sequence is selected from Human CD1a (MLFLLLPLLAVLPGDG; SEQ ID NO:1); Human CD1b (MLLLPFQLLAVLFPGGN; SEQ ID NO:2); Human CD1c (MLFLQFLLLALLLPGGD; SEQ ID NO:3); Human CD1d (MGCLLFLLLWALLQAWGSA; SEQ ID NO:4); Human CD1e (MLLLFLLFEGLCCPGENTA; SEQ ID NO:5); Human LDLR (MGPWGWKLRWTVALLLAAAGT; SEQ ID NO:6); or Human LRP1 (MLTPPLLLLLPLLSALVAA; SEQ ID NO:7). According to this disclosure, signal sequences may be derived from any protein. Signal sequences may range from 4-50 amino acids and may be chimeric, tandem, repeated or inverted. Signal sequences may include those taught herein or any signal sequence which is at least 50, 60, 70, 80, 90, 95 or 99% identical to those taught herein, as long as the signaling function is substantially retained.

In some implementations the transmembrane domain sequence is selected from

Human CD1a
(GFIILAVIVPLLLLIGLALWF; SEQ ID NO: 8);
Human CD1b
(IVLAIIVPSLLLLLCLALWYM; SEQ ID NO: 9);
Human CD1c
(NWIALVVIVPLVILIVLVLWF; SEQ ID NO: 10);
Human CD1d
(MGLIALAVLACLLFLLIVGFT; SEQ ID NO: 11);
Human CD1e
(SIFLILICLTVIVTLVILVVV; SEQ ID NO: 12);
Human LDLR
(ALSIVLPIVLLVFLCLGVFLLW; SEQ ID NO: 13);
or
Human LRP1
(HIASILIPLLLLLLLVLVAGVVFWY; SEQ ID NO: 14).

According to this disclosure, transmembrane domain sequences may be derived from any protein. Transmembrane sequences may range from 10-100 amino acids and may be chimeric, tandem, repeated or inverted. Transmembrane sequences may include those taught herein or any transmembrane sequence which is at least 50, 60, 70, 80, 90, 95 or 99% identical to those taught herein, as long as the function is substantially retained.

In some implementations the cytoplasmic domain sequence is selected from Human CD1a (RKRCFC; SEQ ID NO:15); Human CD1b (RRRSYQNIP; SEQ ID NO:16); Human CD1c (KKHCSYQDIL; SEQ ID NO:17); Human CD1d (SRFKRQTSYQGVL; SEQ ID NO:18); Human CD1e (DSRLKKQSSNKNILSPHTPSPVFLMGANTQDTKNSRHQFCLAQVSWIK-NRVLKKWKTRLNQLW; SEQ ID NO:19); Human LDLR (KNWRLKNINSINFDNPVYQ-KTTEDEVHICHNQDGYSYPSRQMVSLEDDVA; SEQ ID NO:20); and Human LRP1 (KRRVQGAKGFQHQRMTNGAMNVEIGNPTYKMYEGGEPDDVGGLLDADFALDPDKPTNF TNPVYATLYMGGHGSRHSLASTDEKRELLGRGPEDEIGDPLA; SEQ ID NO:21). According to this disclosure, cytoplasmic domain sequences may be derived from any protein. Cytoplasmic sequences may range from 10-100 amino acids and may be chimeric, tandem, repeated or inverted. Cytoplasmic sequences may include those taught herein or any cytoplasmic sequences which are at least 50, 60, 70, 80, 90, 95 or 99% identical to those taught herein, as long as the function is substantially retained.

It is noted that the CD1e sequence structure also contains a N-terminal propeptide sequence (APQALQSYHLAA; SEQ ID NO:22) that is processed in endosomal compartments and is responsible for membrane association, while its absence results in a soluble molecule.

The NCBI reference for each of the above-referenced parental receptor molecules is provided in Table 2.

TABLE 2
Reference Sequences

| PROTEIN ID | NCBI mRNA Reference Sequence |
|---|---|
| Human CD1a | NM_001320652.2 |
| Human CD1b | NM_001764.3 |
| Human CD1c | NM_001765.3 |
| Human CD1d | NM_001319145.2 |
| Human CD1e | NM_001042583.3 |
| Human LDLR | NM_000527.5 |
| Human LRP1 | NM_002332.3 |

### Antigenic Payloads

The vaccine scaffolds of the present disclosure are engineered such that they may be loaded with or have incorporated therein at least one antigenic payload, such as an mRNA encoding an antigenic polypeptide or antigenic peptide. Once an antigenic payload is combined with a vaccine scaffold, the construct may be referred to as a vaccine cassette.

Various diseases and/or conditions may be treated with the pharmaceutical compositions, e.g., vaccines of the present disclosure where the vaccine cassettes include one or more antigenic payloads comprising an mRNA encoding an antigenic polypeptide or antigenic peptide, for which an immune response is desired. Such diseases include cancer, autoimmune diseases and inflammation.

mRNA Nanoparticle Compounds and Compositions

This disclosure provides delivery vehicle molecules encapsulating mRNAs to form mRNA nanoparticles that comprise multicomponent lipid compositions, e.g., multicomponent lipid nanoparticles, comprising complexes of lipidated cationic peptide compounds in combination with further lipid components, such as structural lipids, phospholipids, and shielding lipids. The multicomponent lipid compositions and complexes may include cationic peptide-phospholipid conjugates, known as lipitoids, or N-substituted cationic peptide compounds that are N-substituted with lipid moieties and/or (oligo- and/or poly)ethylene glycol moieties (referred to herein as tertiary amino lipidated and/or PEGylated cationic peptide compounds) as the cationic peptide compounds.

The multicomponent lipid compositions comprising lipitoids and/or tertiary amino lipidated and/or PEGylated cationic peptide compounds of the delivery vehicles exhibit highly efficient delivery of encapsulated mRNAs into cells that exceeds the efficiency observed for other commercially available lipid nanoparticle formulations (such as formulations found in commercially available kits) or standalone agents. Interestingly, delivery vehicles having lipid compositions of this disclosure still achieved successful delivery of mRNAs without the inclusion of structural lipids.

This disclosure provides compositions comprising complexes, wherein the complexes comprise one or more polyanionic compounds and three or more lipid components. In some implementations of the foregoing, the complexes comprise one or more polyanionic compounds, one or more lipidated cationic peptide compounds, and two or more other lipid components.

In some implementations wherein the compositions and complexes comprising one or more lipidated cationic peptide compounds with one or more polyanionic compounds and/or non-anionic compounds further comprise two or more lipid components (such as the phospholipids, structural lipids, and/or shielding lipids described herein), the compositions may be described as a lipid formulation. In certain implementations wherein the composition comprises two or more lipid components, the lipid composition may be described as a multicomponent lipid formulation. In certain implementations wherein the complex comprises a lipidated cationic peptide compound, a polyanionic compound, a phospholipid, a structural lipid, and a shielding lipid, the composition comprises a lipid nanoparticle formed from a delivery vehicle encapsulating at least one mRNA according to the disclosure. In still other implementations wherein the composition comprises a lipid nanoparticle complex formed of a delivery vehicle encapsulating at least one mRNA according to the disclosure, the composition may be characterized as a lipid nanoparticle (LNP) composition.

In some implementations, the compositions of this disclosure comprise complexes of one or more polyanionic compounds, and lipid components, wherein the lipid components comprise optionally one or more structural lipids; one or more phospholipids, one or more shielding lipids, and one or more lipidated cationic peptide compounds. In some implementations, the compositions of this disclosure comprise complexes of one or more polyanionic compounds, and lipid components, wherein the lipid components comprise one or more phospholipids, one or more shielding lipids, and one or more lipidated cationic peptide compounds. In other implementations, the compositions comprise complexes of one or more polyanionic compounds, and lipid components, wherein the lipid components comprise one or more structural lipids; one or more phospholipids, one or more shielding lipids, and one or more lipidated cationic peptide compounds.

The compositions of this disclosure comprise complexes, wherein the complexes comprise one or more polyanionic compounds, and lipid components, wherein the lipid components comprise optionally one or more structural lipids; one or more phospholipids; one or more shielding lipids; and one or more lipidated cationic peptide compounds.

By virtue of their positive charge, the lipidated cationic peptide compounds in the complexes and composition can form complexes with counterbalance the negative charge on the polyanionic cargoes, thus promoting uptake of the cargoes into the target cell. The lipidated cationic peptide compounds as described herein have a net zero charge or a net positive charge. In some implementations wherein the complex or composition comprises one or more cationic compounds, the one or more cationic compounds independently have a net zero charge or a net positive charge. In certain implementations, the one or more lipidated cationic peptide compounds independent have a net positive charge of at least +1. It should be recognized that the net charge present on the one or more cationic compounds may vary depending upon environmental conditions. For example, in some implementations, the one or more cationic compounds independently have a stable, net positive charge at physiologically relevant pH ranges. For example, physiological pH is at least about 5.5 and typically at least about 6.0. More typically, physiological pH is at least about 6.5. Usually, physiological pH is less than about 8.5 and typically less than about 8.0. More typically, physiological pH is less than about 7.5.

The lipidated cationic peptide compounds utilized in the compositions as provided herein may include cationic peptoid-phospholipid conjugate constructs, also known as lipitoids, or N-substituted cationic peptide compounds possessing lipid moieties and/or (oligo- and/or poly)ethylene glycol moieties throughout the peptide backbone, herein referred to as tertiary amino lipidated and/or PEGylated cationic peptide compounds. In some implementations, the compositions of this disclosure comprise complexes comprising one or more tertiary amino lipidated and/or PEGylated cationic peptide compounds. In other implementations, the compositions comprise complexes comprising one or more lipitoids. In still other implementations, the compositions comprise complexes comprising one or more tertiary amino lipidated and/or PEGylated cationic peptide compounds, one or more lipitoids, or any combinations thereof.

In some implementations, the complexes and compositions of this disclosure comprise one or more tertiary amino lipidated and/or PEGylated cationic peptide compounds. The tertiary amino lipidated and/or PEGylated cationic peptide compounds are peptide chains comprising N-substituted amino acid residues. The tertiary amino lipidated and/or PEGylated cationic peptide compounds of this disclosure comprise an oligopeptide backbone, wherein the oligopeptide backbone comprises repeating subunits of N-substituted cationic amino acid residues optionally interleaved with N-substituted neutral (“spacer”) and/or lipid amino acid residues. The oligopeptide backbone is further capped at the N- and/or C-terminus by amino acid residues that are N-substituted with lipid moieties (“N-lipidated”) and/or N-substituted with oligoethylene glycol and/or polyethylene glycol (“N-PEGylated”).

In some implementations, the tertiary amino lipidated and/or PEGylated cationic peptide compound may be characterized by a total number of amino acid residues present in the peptide compound, wherein each amino acid residue is represented by the general structure —(NR—CRaRb—C(O))—. In some implementations, the total number of amino acid residues is between 2 and 40 amino acid residues, between 2 and 30 amino acid residues, between 3 and 25 amino acid residues, between 5 and 20 amino acid residues, or between 7 and 15 amino acid residues. In certain implementations, the tertiary amino lipidated and/or PEGylated cationic peptide compound comprises between 5 and 20 amino acid residues.

In other implementations, the tertiary amino lipidated and/or PEGylated cationic peptide compound has a net zero charge or a net positive charge. In certain implementations wherein the tertiary amino lipidated and/or PEGylated cationic peptide compound is a tertiary amino lipidated cationic peptide compound, the tertiary amino lipidated cationic peptide compound has a net positive charge of at least +1. In other implementations wherein the tertiary amino lipidated and/or PEGylated cationic peptide compound is a tertiary amino PEGylated cationic peptide compound or a tertiary amino lipidated and PEGylated cationic peptide compound, the cationic peptide compound has a net zero charge (i.e., is charge neutral) or a net positive charge. In some implementations wherein the tertiary amino lipidated and/or PEGylated cationic peptide compound is a tertiary amino PEGylated cationic peptide compound or a tertiary amino lipidated and PEGylated cationic peptide compound, the cationic peptide compound has a net positive charge of +1. In certain implementations, the tertiary amino lipidated and/or PEGylated cationic peptide compound has a net positive charge of (rxp)+.

The tertiary amino lipidated and/or PEGylated cationic peptide compounds may be useful for complexation with polyanionic compounds, such as mRNAs, and for the delivery of such polyanionic compounds into cells. The tertiary amino lipidated and/or PEGylated cationic peptide compounds of this disclosure comprise an oligopeptide backbone of repeating subunits of N-substituted cationic amino acid residues optionally interleaved with N-substituted neutral spacer amino acid residues and/or N-lipidated amino acid residues.

The cationic amino acid residues in the repeating subunits of the oligopeptide backbone confer positive charge to the compounds of this disclosure, which allows for favorable electrostatic interaction with and charge neutralization of polyanionic species like mRNAs. The interleaving of neutral or lipidated amino acid residues in between the cationic residues allows for greater control over the spatial distribution of positive charge throughout the tertiary amino lipidated and/or PEGylated cationic peptide compounds, which enables improved complexation of the cationic peptide compounds to polyanionic species having specific lengths, charge distributions and/or conformations.

Each repeating subunit of the tertiary amino lipidated and/or PEGylated cationic peptide compounds of this disclosure comprises at least one cationic amino acid residue. The cationic amino acid residues provide the positive charge that enables the peptide compounds described herein to form electrostatic complexes with nucleic acids or other polyanionic compounds, by interaction with negative charges on the nucleic acids or polyanionic compounds. Complexation of nucleic acids partially or fully shields the negative charge of the nucleic acid and facilitates transport through the lipid membrane of cells and into the cell interior.

The tertiary amino lipidated and/or PEGylated cationic peptide compounds described herein may comprise multiple cationic moieties along the oligopeptide backbone in close proximity to one another. When multiple cationic moieties are present along the oligopeptide backbone, the protonated or deprotonated state of certain cationic moieties may influence the pKa values of other cationic moieties in close proximity.

Cationic, or positively charged, moieties may include, for example, nitrogen-based substituents, such as those containing the following functional groups: amino, guanidino, hydrazido, and amidino. These functional groups can be either aromatic, saturated cyclic, or aliphatic.

Within the oligopeptide backbone of tertiary amino lipidated and/or PEGylated cationic peptide compounds, the cationic amino acid residues may be optionally interleaved with neutral spacer amino acid residues, possessing a neutral spacer moiety at the N-position. The neutral amino acid residues may be useful to modulate the spatial distribution of the positive charge in the tertiary amino lipidated and/or PEGylated cationic peptide compounds for improved electrostatic interactions with the polyanionic compounds, including polynucleotides, to be complexed with the cationic peptide compounds. Neutral spacer moieties may include any substituents that are neutral, or have zero net charge, at physiologically relevant pH ranges.

In addition to the optional interleaving of neutral amino acid residues with cationic amino acid residues within the oligopeptide backbone of tertiary amino lipidated and/or PEGylated cationic peptide compounds, N-lipidated amino acid residues, possessing a lipid moiety at the N-position, may also optionally be interleaved with the cationic (and optional neutral spacer) amino acid residues. In some implementations wherein the tertiary amino lipidated and/or PEGylated cationic peptide compound comprises N-lipidated amino acid residues, the tertiary amino lipidated and/or PEGylated cationic peptide compound is N-lipidated. Similar to the neutral amino acid residues, the N-lipidated amino acid residues within the oligopeptide backbone may be useful to modulate the spatial distribution of the positive charge in the tertiary amino lipidated and/or PEGylated cationic peptide compounds as well as augment their lipophilicity for improved encapsulation of polyanionic materials and endocellular delivery. The spacing of lipids along the peptoid backbone may also influence the lipid fluidity/crystallinity which is known to influence cellular uptake and endosomal release. Suitable lipid moieties may include, for example, optionally substituted branched or straight chain aliphatic moieties, or optionally substituted moieties derived from natural lipid compounds, including fatty acids, sterols, and isoprenoids.

In some implementations, the lipid moieties may include branched or straight chain aliphatic moieties having from about 6 to about 50 carbon atoms or from about 10 to about 50 carbon atoms, optionally comprising one or more heteroatoms, and optionally comprising one or more double or triple bonds (i.e., saturated or mono- or poly-unsaturated). In certain implementations, the lipid moieties may include optionally substituted aliphatic, straight chain or branched moieties, each hydrophobic tail independently having from about 8 to about 30 carbon atoms or from about 6 to about 30 carbon atoms. In certain implementations, the lipid moieties may include, for example, aliphatic carbon chains derived from fatty acids, fatty alcohols, phospholipids, glycerides (such as di- or tri-glycerides), glycosylglycerides, sphingolipids, ceramides, and saturated and unsaturated sterols, and isoprenoids. Other suitable lipid moieties may include lipophilic carbocyclic or aromatic groups such as optionally substituted aryl, cycloalkyl, cycloalkylalkyl, or arylalkyl moieties, including for example naphthalenyl or ethylbenzyl, or lipids comprising ester functional groups including, for example, sterol esters and wax esters.

The tertiary amino lipidated and/or PEGylated cationic peptide compounds of this disclosure comprise an oligopeptide backbone that is capped at its N- and/or C-terminus by amino acid residues that are N-substituted with lipid moieties (“N-lipidated”) and/or N-substituted with oligoethylene glycol and/or polyethylene glycol (“N-PEGylated”). The incorporation of N-lipidated amino acid residues at the N- and/or C-terminus of the cationic peptide compounds described herein increase the lipophilicity of the compounds. The increased lipophilicity of the cationic peptide compounds enhances their affinity for hydrophobic environments, such as the lipid bilayer of the cell membrane, thus increasing the propensity of the tertiary amino lipidated and/or PEGylated cationic peptide compounds, and any complexes thereof with polyanionic compounds, to be transported into the cell.

The tertiary amino lipidated and/or PEGylated cationic peptide compounds of this disclosure may comprise amino acid residues that are N-lipidated and/or N-PEGylated. The cationic peptide compounds provided herein comprise at least one amino acid residue that is N-lipidated or N-PEGylated. The tertiary amino lipidated and/or PEGylated cationic peptide compounds of this disclosure also may be provided with the N-terminus and C-terminus in their free amine and free acid forms, respectively.

In certain implementations, the complex or composition may comprise a combination of one or more tertiary amino lipidated and/or PEGylated cationic peptide compounds which are cation-rich with one or more tertiary amino lipidated and/or PEGylated cationic peptide compounds which are highly lipidated and/or contain at least one PEG moiety. In theory, such a combination could confer improved delivery of polyanionic compounds by providing greater charge stabilization (via the cation-rich peptide compounds) along with greater lipophilic shielding (by virtue of the lipidated peptide compounds). It should further be recognized that the individual amounts of each of the individual tertiary amino lipidated and/or PEGylated cationic peptide compounds may be adjusted to achieve the desired properties.

The tertiary amino lipidated and/or PEGylated cationic peptides may be synthesized entirely by methods known in the art for producing N-substituted residues in a peptide chain, using either or both solid-phase and solution-phase methods of synthesis.

In some implementations, the one or more lipidated cationic peptide compounds of the complexes and compositions described herein comprises one or more cationic peptoid-phospholipid conjugate constructs, also known as lipitoids. Lipitoids are N-substituted polyglycine compounds (also known as “peptoids”) having a combination of cationic and/or neutral side chains at the N-positions of glycine residues along peptoid backbone, which are further conjugated to a single terminal phospholipid group of the peptoid chain. Lipitoids and methods for their synthesis are known in the art.

Cationic, or positively charged, moieties may include, for example, nitrogen-based substituents, such as those containing the following functional groups: amino, guanidino, hydrazido, and amidino. These functional groups can be either aromatic, saturated cyclic, or aliphatic. In some implementations of the lipitoid, each cationic moiety is independently aminoalkyl, alkylaminoalkyl, aminoalkylaminoalkyl, guanidinoalkyl, or N-heterocyclylalkyl.

Neutral moieties may include but are not limited to a C₁-C₄-alkyl substituted by cycloalkyl, heterocyclylalkyl, alkylaryl, arylalkyl, alkylheteroaryl, heteroarylalkyl, alkoxy, alkoxyalkyl, or hydroxyalkyl, wherein each cycloalkyl, heterocyclylalkyl, alkylaryl, arylalkyl, alkylheteroaryl, heteroarylalkyl, alkoxy, alkoxyalkyl, or hydroxyalkyl is optionally substituted with one or more substituents —OH, halo, or alkoxy.

Similar to the tertiary amino lipidated and/or PEGylated cationic peptide compounds, the lipitoids described herein may include the amino acid residues arranged in random sequences, or repeating motifs in alternating sequences or block sequences.

In addition to the lipidated cationic peptide compounds employed as the primary lipid components for charge neutralization of the one or more polyanionic compounds, such as mRNAs, as cargo, the multicomponent lipid compositions and complexes therein may comprise other lipid components including structural lipids, phospholipids and shielding lipids. The additional lipid components described herein provide physical structure and stability to the complex of the cationic peptide compounds and polyanionic material that facilitate their administration in solution and promote the uptake of the complexes into the cell.

Structural lipids may be used as described herein to confer physical stability to the complexes of the polyanionic compounds within the multicomponent compositions, and enhance lipophilic character of the complexes to promote binding and endocytosis with the target cells. In some implementations, the compositions of this disclosure comprise complexes comprising optionally one or more structural lipids as lipid components. In some variations, the compositions comprise complexes comprising one or more structural lipids.

When present, suitable structural lipids for the compositions and complexes of this disclosure may include but are not limited to sterols. In some implementations, the structural lipid is selected from the group consisting of cholesterol, fecosterol, sitosterol, ergosterol, campesterol, stigmasterol, brassicasterol, tomatidine, ursolic acid, alpha-tocopherol, and mixtures thereof. In certain implementations, the structural lipid is cholesterol. In some implementations of this disclosure, the compositions and complexes do not contain structural lipids, but still exhibit very good delivery efficiency.

Phospholipids, like structural lipids, may also be incorporated into the complexes and compositions of this disclosure. Phospholipids provide further stabilization to complexes in solution as well as facilitate cell endocytosis, by virtue of their amphipathic character and ability to disrupt the cell membrane. In some implementations, the compositions provided herein comprises complexes comprising one or more phospholipids as lipid components. In certain implementations, the one or more phospholipids comprise one or more zwitterionic phospholipids.

In some implementations, the one or more phospholipids are selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C 16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-dipalmitoyl-sn-glycero-3-phosphoethanolamine (DPPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof. In certain implementations, the phospholipid is DOPE.

It should be recognized that the amphipathic attributes provided by the phospholipids to facilitate the disruption of the cellular membrane for endocytosis may be alternatively provided by zwitterionic lipids that are not phospholipids. In some implementations, the compositions comprise complexes comprising one or more zwitterionic lipids that are not phospholipids. In still further implementations, the comprises comprise complexes comprising one or more phospholipids, one or more zwitterionic lipids, or any combinations thereof.

The complexes and compositions of this disclosure may further include one or more shielding lipids. Shielding lipids, such as PEGylated lipids, may provide an additional layer of charge neutralization to the one or more lipidated cationic peptide compounds as countercharge to the one or more polyanionic compounds and prevent clearance by the cellular phagocytic processes. In some implementations, the compositions provided herein comprises complexes comprising one or more shielding lipids as lipid components.

In some implementations, the shielding lipid is a PEG lipid. In other implementations, the PEG lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof. In some implementations, the PEG lipid is a PEG-modified phosphatidylethanol selected from the group consisting of PEG-modified DSPE (DSPE-PEG), PEG-modified DPPE (DPPE-PEG), and PEG-modified DOPE (DOPE-PEG). In certain implementations, the PEG lipid is selected from the group consisting of dimyristoylglycerol-polyethylene glycol (DMG-PEG), distearoylglycerol-polyethylene glycol (DSG-PEG), dipalmitoylglycerol-polyethylene glycol (DPG-PEG), and dioleoylglycerol-polyethylene glycol (DOG-PEG). In certain implementations, the PEG lipid is DMG-PEG.

Molecular weights of the PEG chain in the foregoing PEG lipids may be especially advantageous for incorporation into the complexes of this disclosure. For example, in some implementations, the PEG chain has a molecular weight between 350 and 6,000 g/mol, between 1,000 and 5,000 g/mol, or between 2,000 and 5,000 g/mol. In certain implementations, the PEG chain of the PEG lipid has a molecular weight of about 350 g/mol, 500 g/mol, 600 g/mol, 750 g/mol, 1,000 g/mol, 2,000 g/mol, 3,000 g/mol, 5,000 g/mol, or 10,000 g/mol. In certain other implementations, the PEG chain of the PEG lipid has a molecular weight of about 500 g/mol, 750 g/mol, 1,000 g/mol, 2,000 g/mol or 5,000 g/rmol. For example, in certain implementations, the PEGylated lipid is dimyristoylglycerol-polyethylene glycol 2000 (DMG-PEG 2000).

In still further implementations, the one or more PEG lipids comprise a tertiary amino PEGylated cationic peptide compounds of formula (I) comprising at least one oligo- or polyethylene glycol moiety. It should be recognized that the tertiary amino lipidated and/or PEGylated cationic peptide compounds of formula (I) may comprise several short oligoethylene glycol moieties in lieu of fewer longer polyethylene glycol moieties and provide similar particle stabilization to the complex.

Multicomponent lipid compositions comprising lipidated cationic peptide compounds of this disclosure may be useful for complexation with one or more polyanionic compounds, such as mRNAs. In one aspect, this disclosure provides compositions comprising complexes comprising one or more mRNAs complexed to one or more lipidated cationic peptide compounds and two or more other lipid components.

In some implementations, the one or more mRNAs may be naturally occurring or not naturally occurring variations with unnatural backbone and modified backbone linkages such as phosphorothioate, unnatural and modified bases, and unnatural and modified termini. The mRNAs may be recombinantly produced or chemically synthesized molecules.

The mRNA encodes an antigenic peptide or an antigenic polypeptide, and antigenic polypeptides may comprise one or more antigenic peptides. In some implementations, the combined delivery of two or more particular mRNAs together may be especially useful for the immunogenic methods of this disclosure.

The compositions of this disclosure comprise complexes comprising one or more mRNAs, and lipid components, wherein the lipid components comprise one or more lipidated cationic peptide compounds, one or more phospholipids, one or more shielding lipids, and optionally one or more structural lipids. The physical properties of the complexes and compositions described herein may be influenced by the particular selection of lipid components for a given polyanionic compound as well as by the quantities of each component within the complexes and compositions. In some implementations, the lipid components within the complexes and compositions thereof may be characterized by the mass percentages of the lipid components (alone or in combination) with respect to mass of the total lipid components present and/or mass ratios of individual lipid components with respect to one another.

Additional components may also be added to the complexes to facilitate high encapsulation of mRNA polyanionic cargoes and/or targeted, controlled release thereof. Such additional components may include, for example, polymers and surface-active components.

The complexes may include further components useful for delivery of mRNA cargoes and other compounds to cells. Such components may include but are not limited to those which together form, for example, supercomplexes or other delivery systems (e.g., hybrid lipid-polymer nanoparticles) comparable to delivery vehicles encapsulating mRNAs to form lipid mRNA nanoparticles.

The incorporation of polymers into the complexes described herein may stabilize the complexes comprising the lipidated cationic peptide compounds and polyanionic compounds by forming delivery vehicles encapsulating mRNAs to form polymeric mRNA nanoparticle vesicles, or in the presence of additional lipid components, hybrid lipid-polymer mRNA nanoparticles. In some implementations, the complexes of this disclosure comprise polymers. Suitable polymers may include neutral polymers (such as poly(lactic-co-glycolic acid) (PLGA) or polyglycolic acid (PGA)), anionic polymers (including poly(aspartate), poly(glutamate), and heparin), and/or cationic polymers (e.g., polyethyleneimine, protamine).

Additional description of compositions and formulations of nanoparticles and their mRNA cargoes, along with descriptions of methods of producing such compositions and preparing such formulations, is provided in International Patent Application No. PCT/US19/053661, PCT/US19/053655, and NL 2026825, each of which is incorporated herein in its entirety, as noted above.

Organic materials are contemplated for use in preparing the delivery vehicle molecules encapsulating mRNAs to form lipitoid mRNA nanoparticles of this disclosure. mRNA nanoparticle polymers include polystyrene, silicone rubber, polycarbonate, polyurethanes, polyalkanes including polyethylene and polypropylene, polymethylmethacrylate, polyvinyl chloride, polyesters, and polyethers. Biodegradable biopolymers (e.g., polypeptides such as BSA, polysaccharides, and the like), other biological materials (e.g., carbohydrates), and/or polymeric compounds are also contemplated for use in producing delivery vehicle molecules encapsulating mRNAs to form mRNA nanoparticles.

Liposomal mRNA nanoparticles formed of delivery vehicle molecules encapsulating mRNAs are also contemplated for use in the materials and methods of this disclosure. Hollow nanoparticles are also contemplated. Liposomal mRNA nanoparticles formed of delivery vehicle molecules encapsulating mRNAs of this disclosure have at least a substantially spherical geometry, an internal face and an external face, and comprise a lipid bilayer. The lipid bilayer comprises, in various implementations, a lipid from the phosphocholine family of lipids or the phosphoethanolamine family of lipids. While not meant to be limiting, the lipid found in delivery vehicle molecules encapsulating mRNAs to form liposomal mRNA nanoparticles of this disclosure may be 1,2-dioleoyl-sn-glycero-3-phosphocholine (DOPC), 1,2-dimyristoyl-sn-phosphatidylcholine (DMPC), 1-palmitoyl-2-oleoyl-sn-phosphatidylcholine (POPC), 1,2-distearoyl-sn-glycero-3-phospho-(1′-rac-glycerol) (DSPG), 1,2-dioleoyl-sn-glycero-3-phospho-(1′-rac-glycerol) (DOPG), 1,2-distearoyl-sn-glycero-3-phosphocholine (DSPC), 1,2-dipalmitoyl-sn-glycero-3-phosphocholine (DPPC), 1,2-di-(9Z-octadecenoyl)-sn-glycero-3-phosphoethanolamine (DOPE), 1,2-dihexadecanoyl-sn-glycero-3-phosphoethanolamine (DPPE), cardiolipin, lipid A, or a combination thereof.

The use of mixtures of particles having different sizes, shapes and/or chemical compositions, as well as the use of delivery vehicle molecules encapsulating mRNAs to form mRNA nanoparticles having uniform sizes, shapes and chemical composition, are contemplated. Examples of suitable particles include, without limitation, delivery vehicle molecules encapsulating mRNAs to form mRNA nanoparticles wherein the nanoparticle component is of conventional structure, aggregate particles, isotropic (such as spherical particles) and anisotropic particles (such as non-spherical rods, tetrahedral, prisms), and core-shell particles.

The delivery vehicle molecules encapsulating mRNAs to form mRNA nanoparticles comprise materials described herein that may be available commercially or can be produced from progressive nucleation in solution (e.g., by colloid reaction), or by various physical and chemical vapor deposition processes, such as sputter deposition.

The delivery vehicle molecules encapsulating mRNAs to form mRNA nanoparticles can range in size from about 1 nm to about 250 nm in mean diameter, about 1 nm to about 240 nm in mean diameter, about 1 nm to about 230 nm in mean diameter, about 1 nm to about 220 nm in mean diameter, about 1 nm to about 210 nm in mean diameter, about 1 nm to about 200 nm in mean diameter, about 1 nm to about 190 nm in mean diameter, about 1 nm to about 180 nm in mean diameter, about 1 nm to about 170 nm in mean diameter, about 1 nm to about 160 nm in mean diameter, about 1 nm to about 150 nm in mean diameter, about 1 nm to about 140 nm in mean diameter, about 1 nm to about 130 nm in mean diameter, about 1 nm to about 120 nm in mean diameter, about 1 nm to about 110 nm in mean diameter, about 1 nm to about 100 nm in mean diameter, about 1 nm to about 90 nm in mean diameter, about 1 nm to about 80 nm in mean diameter, about 1 nm to about 70 nm in mean diameter, about 1 nm to about 60 nm in mean diameter, about 1 nm to about 50 nm in mean diameter, about 1 nm to about 40 nm in mean diameter, about 1 nm to about 30 nm in mean diameter, or about 1 nm to about 20 nm in mean diameter, or about 1 nm to about 10 nm in mean diameter. In other aspects, the size of the mRNA nanoparticles is from about 5 nm to about 150 nm (mean diameter), from about 5 to about 50 nm, from about 10 to about 30 nm, from about 10 to 150 nm, from about 10 to about 100 nm, or about 10 to about 50 nm. The size of the mRNA nanoparticles may be from about 5 nm to about 150 nm (mean diameter), from about 30 to about 100 nm, from about 40 to about 80 nm. The size of the mRNA nanoparticles used in a method of this disclosure may vary, as required by their particular use or application. The variation of size is advantageously used to optimize certain physical characteristics of the mRNA nanoparticles.

Apparent from the foregoing description of delivery vehicle molecules encapsulating mRNAs to form mRNA nanoparticles is that a wide variety of mRNA nanoparticles are contemplated for use in delivering one or more mRNAs to cells of a subject in need of prophylaxis or treatment of a disease such as cancer, including solid tumor forms of cancer, an autoimmune disease, or inflammation.

The following examples provide data establishing methods of preparing mRNA nanoparticles comprising delivery vehicle molecules encapsulating therein mRNA encoding at least one antigenic peptide useful in recruiting a diseased subject's immune system to provide a personalized medical approach to the treatment of diseases such as cancer (e.g., tumors).

### NON-LIMITING WORKING EXAMPLES

### Example 1

### PBMC Transfection Efficacy

Peripheral blood mononuclear cell transfection efficacy was assessed using cryopreserved healthy donor (HD) peripheral blood mononuclear cells that were thawed and resuspended in 14 mL RPMI1640. Cells were pelleted by centrifugation at 1200 rpm for 10 minutes. Supernatant was aspirated and cells were re-suspended and counted in appropriate volumes of culture media (1:1 AIM-V/RPMI 1640+10% filtered human AB Serum+50 μM β-mercaptoethanol (TC grade)). Cells were rested overnight at 37° C. in a CO₂ incubator (5% CO₂). After incubation cells were treated with 100 ng mRNA encoding rat Thy1.1 encapsulated in delivery vehicles to form mRNA nanoparticles in 50 μl culture media. Cells were incubated at 37° C. in CO₂ incubator (5% CO₂) for 24 hours. After 24 hours, cells were harvested and washed twice in Phosphate buffered saline (PBS) pH 7.2. Washed cells were then stained with Zombie Near Infrared live dead stain (NIR)(BioLegend) in PBS for 15 minutes at room temperature (RT). Cells where then washed and re-suspended in 100 μl FACS buffer (PBS+0.5%BSA+0.02% sodium azide) containing fluorochrome conjugated anti-rat Thy1.1, anti-CD8 antibody (i.e., a-CD8), a-CD4, a-CD56, a-CD11c, a-CD19, a-MHC class II, and a-CD14 (BioLegend). Cells were then incubated for 20 minutes at room temperature. After staining, cells were washed twice with 200 μl PBS followed by 10 minutes centrifugation at 1200 rpm. After the final wash, supernatant was discarded, and cells were resuspended in 200 μl PBS. Resuspended cells were then analyzed on a flow cytometer (Cytek).

The delivery vehicle-encapsulated mRNA delivered cargo to primary immune cells from healthy donor PBMCs. Using the delivery vehicles encapsulating mRNAs to form mRNA nanoparticles, we observed high transfection efficacy in the APC populations: B-cells, DCs, and monocytes, as well as primary T cells and NK cells. See FIG. 4.

This experiment establishes the efficient delivery and translation of mRNA cargo in professional antigen presentation cell populations mitigates, and thereby effectively eliminates, the need for APC isolation and in vitro differentiation. Protein expression in these cell types enhances antigen presentation and subsequent T cell stimulation, establishing these cell types as advantageous in the immunotherapeutic methods of the disclosure.

### Example 2

APCs Comprising mRNA Encoding Antigenic Peptide

Antigen presenting cells harboring mRNAs encoding antigen peptides delivered by delivery vehicles encapsulating mRNAs to form mRNA nanoparticles were analyzed for their antigen presentation and T cell activation properties. Cryopreserved human cytomegaly virus (CMV) sero-positive healthy donor (CMV+) peripheral blood mononuclear cells were thawed and resuspended in 14 mL RPMI1640. Cells were pelleted by centrifugation at 1200 rpm for 10 minutes. Supernatant was aspirated and cells were re-suspended and counted in an appropriate volume of culture media (1:1 AIM-V/RPMI 1640+10% filtered human AB Serum+50 μM β-mercaptoethanol (TC grade)). Cells were rested overnight at 37° C. in a CO₂ incubator (5% CO₂). After incubation, cells were treated with 50 ng mRNA encoding native CMV pp65 protein, 2 μg/mL CMV pp65 peptide pool covering the entire pp65 molecule, or non-coding mRNA. Cells were incubated at 37° C. in CO₂ incubator (5% CO₂) for 24 hours. After 24 hours, cells and cell culture supernatant were harvested and cells were washed twice in phosphate-buffered saline (PBS) pH 7.2. Washed cells were then stained with Zombie Near Infrared live dead stain (NIR)(BioLegend) in PBS for 15 minutes at room temperature (RT). Cells where then washed and re-suspended in 100 μl FACS buffer (PBS+0.5%BSA+0.02% sodium azide) containing fluorochrome conjugated anti-CD8 antibody (i.e., a-CD8), a-CD4, a-CD137, and a-CD69 (BioLegend). Cells were then incubated for 20 minutes at room temperature. After staining, cells were washed twice with 200 μl PBS followed by 10 minutes centrifugation at 1200 rpm. After the final wash, supernatant was discarded, and cells were resuspended in 200 μl PBS. Resuspended cells were then analyzed on a flow cytometer (Cytek). Supernatant was used for measuring secreted Interferon gamma (IFNγ) using standardized commercially available human IFNγ ELISA kits and protocol (Thermo Scientific).

Experimental results are presented in FIG. 5. FIG. 5A provides the results of flow cytometry of CD8 T cell activation after antigen challenge. FIG. 5B shows IFNγ secretion in antigen-treated or control-treated PBMC samples. Upon recognition of their cognate antigen, CD8 T cell upregulate surface expression of proteins often referred to as activation markers. Activated CD8 T cells also produce and secrete IFNγ. Thus, secreted IFNγ and the expression of activation markers serve as identifiers of antigen-specific CD8 T cell responses. Two such activation markers specific for CD8 T cells that have recognized antigen presented on HLA molecules are CD69 and CD137. We observed robust CD8 T cell activation both by using peptide antigens and mRNA encoded antigens. Unexpectedly and therefore strikingly, antigen delivered in mRNA form gave a stronger response by means of more CD8 T cells expressing activation markers and more IFNγ secreted into the supernatant than antigen delivered in peptide form.

Although both peptide and mRNA treatments theoretically cover the same antigens, and despite peptide being available to all APCs in the PBMC population while mRNA only gets into a subset of the APCs, we observed better responses when the cells use their endogenous machinery to translate, transport, process, and present the antigens from mRNA delivered to the cells.

### Example 3

Optimizing mRNA Design by Including MHC Presentation-Enhancing Sequence

The design of the mRNA encoding antigenic peptide was optimized by incorporating Major Histocompatibility Complex presentation-enhancing sequence. Cryopreserved human cytomegaly virus (CMV) sero-positive healthy donor (CMV+) peripheral blood mononuclear cells were thawed and resuspended in 14 mL RPMI1640. Cells were pelleted by centrifugation at 1200 rpm for 10 minutes. Supernatant was aspirated and cells were re-suspended and counted in appropriate volumes of culture media (1:1 AIM-V/RPMI 1640+10% filtered human AB Serum+50 μM β-mercaptoethanol (TC grade)). Cells were rested overnight at 37° C. in a CO₂ incubator (5% CO₂). After incubation, cells were treated with 50 ng mRNA encoding native CMV pp65 protein, 50 ng mRNA encoding pp65 with MHC presentation-enhancing sequences, 2 μg/mL CMV pp65 peptide pool covering the entire pp65 molecule, or non-coding mRNA. Cells were incubated at 37° C. in a CO₂ incubator (5% CO₂) for 24 hours. After 24 hours, cells and cell culture supernatant were harvested and cells were washed twice in PBS, pH 7.2. Washed cells were then stained with Zombie Near Infrared live dead stain (NIR) (BioLegend) in PBS for 15 minutes at room temperature (RT). Cells where then washed and re-suspended in 100 μl FACS buffer (PBS+0.5%BSA+0.02% sodium azide) containing fluorochrome conjugated anti-CD8 antibody (i.e., a-CD8), a-CD4, a-CD137, and a-CD69 (BioLegend). Cells were then incubated for 20 minutes at room temperature. After staining, cells were washed twice with 200 μl PBS followed by 10 minutes centrifugation at 1200 rpm. After the final wash, supernatant was discarded, and cells were resuspended in 200 μl PBS. Resuspended cells were then analyzed on a flow cytometer (Cytek). Supernatant was used for measuring secreted Interferon gamma (IFNγ) using standardized commercially available human IFNγ ELISA kits and protocol (Thermo Scientific).

The results, presented in FIG. 7, were similar to the results shown in FIG. 5, but a stronger response was observed in the samples that received the pp65 mRNA with MHC presentation-enhancing sequences. By introducing MHC presentation-enhancing sequences, antigen presentation and CD8 T cell activation were improved in these PBMC samples.

### Example 4

### Activated T Cells Kill Antigen-Expressing Target Cells and Can be Passively Expanded

T cells activated by naturally occurring antigen-presenting cells (i.e., APCs) kill target cells presenting the antigen. In addition, the experimental results establish that these T cells are amenable to passive expansion to reach significant numbers in a cost-effective and straightforward manner. Cryopreserved human cytomegaly virus (CMV) sero-positive healthy donor (CMV+) peripheral blood mononuclear cells were thawed and resuspended in 14 mL RPMI1640. Cells were pelleted by centrifugation at 1200 rpm for 10 minutes. Supernatant was aspirated and cells were re-suspended and counted in appropriate volumes of culture media (1:1 AIM-V/RPMI 1640+10% filtered human AB Serum+50 μM β-mercaptoethanol (TC grade)). Cells were rested over night at 37° C. in a CO₂ incubator (5% CO₂). After incubation, 8×10⁶ cells were treated with either 1 μg mRNA encoding pp65 with MHC presentation-enhancing sequences, 2 μg/mL CMV pp65 peptide pool covering the entire pp65 molecule, or non-coding mRNA. Cells were incubated at 37° C. in a CO₂ incubator (5% CO₂) in culture media without additional cytokines to support T cell growth for 6 days. After 6 days, cells were harvested and CD8 T cells were isolated using a human CD8 isolation kit (STEMCELL). Viability was measured and cells were counted. 50,000 isolated CD8 T cells from either peptide-treated or mRNA treated samples were seeded in 8 replicates in 100 μl complete media in a 96-well U-bottom plate. Next, HLA-A2:01-expressing T2 cells (ATCC) were labelled with cell tracer violet dye according to the manufacturer's protocol (Thermo Scientific). After labelling, cells were washed twice in pre-heated media before viability and cell number were assessed. Next, half of the T2 cells were pulsed with CMV pp65 peptide at 37° C. for 1 hour. The other half of the T2 cells were left unpulsed. After 1 hour, cells were washed 2 times in pre-heated complete media before 10,000 pulsed or unpulsed T2 cells were added to the wells containing the isolated CD8 T cells. Isolated CD8 T cells and pulsed or unpulsed T2 cells were co-incubated for 4 hours at 37° C. After 4 hours, 5 μl propidium iodide (PI) was added to each well and CD8-mediated T2 killing was analyzed by flow cytometry (Cytek).

FIG. 7 presents the results of the experiment. FIG. 7A shows that after 6 days of passive expansion, a robust CD8 T cell growth was observed in cultures treated with 1 μg mRNA encoding pp65 with MHC presentation-enhancing sequences. Both viability and cell numbers were superior to cells treated with peptide. FIG. 7B presents data establishing that the activated and expanded CD8 T cells were able to recognize and kill a T2 target cell only when the T2 target cell was pulsed with CMV-pp65 antigens. Moreover, it is notable that antigen-specific T cells can be passively expanded by exposure to antigenic peptide without providing any other specific treatment, apart from providing an ex vivo or in vivo environment generally compatible with cell viability. As disclosed herein, the methods provided for the passive expansion of T cells from about 2% to over about 80% of lymphocytes, yielding at least about 1.6 million viable T cells. The disclosure thus provides simple, effective and economically advantageous methods capable of transforming the manufacture of T cells in, e.g., personalized medicine applications.

The results observed were surprising and unexpected in establishing the non-intuitive outcome of significantly better killing efficacy in CD8 T cells isolated from cultures that were treated with mRNAs encoding antigenic peptides compared to cultures exposed directly to the antigenic peptides. These results ran contrary to conventional understanding in the field that whether a gene product itself or an expressible mRNA encoding that gene product were provided, the result should be similar in providing functional gene product.

This experiment showed that large numbers of functional (i.e., able to kill target cells) CD8 T cells were generated by treating whole PBMC populations with delivery vehicle molecules encapsulating mRNAs to form mRNA nanoparticles wherein the mRNAs encoded antigenic peptides. This passive expansion protocol shows the robustness of the T cell response brought about by proper antigen processing and presentation accomplished by the cells of the subject being treated.

Each patent or other publication identified herein is expressly incorporated herein by reference in its entirety, as would be apparent to one of ordinary skill in the art from context, the incorporation effectively describing and disclosing, for example, the methodologies described in such publications that might be used in connection with information disclosed herein.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

As used herein, an element or step recited in the singular and preceded with the word “a” or “an” should be understood as not excluding the plural of the elements or steps, unless such exclusion is explicitly stated. Furthermore, references to “one implementation” are not intended to be interpreted as excluding the existence of additional implementations that also incorporate the recited features. Moreover, unless explicitly stated to the contrary, implementations “comprising,” “including,” or “having” an element or a plurality of elements having a particular property may include additional elements whether or not they have that property. Moreover, the terms “comprising,” including,” having,” or the like are interchangeably used herein.

The terms “substantially,” “approximately,” and “about” used throughout this Specification are used to describe and account for small fluctuations, such as due to variations in processing. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%.

There may be many other ways to implement the subject technology. Various functions and elements described herein may be partitioned differently from those shown without departing from the scope of the subject technology. Various modifications to these implementations may be readily apparent to those skilled in the art, and generic principles defined herein may be applied to other implementations. Thus, many changes and modifications may be made to the subject technology, by one having ordinary skill in the art, without departing from the scope of the subject technology. For instance, different numbers of a given module or unit may be employed, a different type or types of a given module or unit may be employed, a given module or unit may be added, or a given module or unit may be omitted.

Underlined and/or italicized headings and subheadings are used for convenience only, do not limit the subject technology, and are not referred to in connection with the interpretation of the description of the subject technology. All structural and functional equivalents to the elements of the various implementations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

What is claimed is:

1. A method comprising:
characterizing polynucleotides from a diseased tissue relative to a control;
identifying candidate mRNAs encoding polypeptides associated with the diseased tissue from the characterized polynucleotides;
encapsulating the mRNAs with at least one delivery vehicle molecule to form at least one mRNA nanoparticle;
introducing the at least one mRNA nanoparticle, wherein the mRNAs encode the polypeptides, to peripheral blood leukocytes or sentinel lymph node leukocytes of the subject;
contacting the peripheral blood leukocytes or sentinel lymph node leukocytes comprising mRNAs with T cells of the subject; and
obtaining at least one antigen-specific T cell.

2. The method according to claim 1 wherein the subject is a human.

3. The method according to any one of claim 1 or 2 wherein the disease is cancer, an infectious disease, an autoimmune disease or inflammation.

4. The method according to any one of claims 1-3 wherein the subject has cancer.

5. The method according to any one of claims 3-4 wherein the cancer is Acute Lymphoblastic Leukemia (ALL), Acute Myeloid Leukemia (AML), Adrenocortical Carcinoma, Kaposi Sarcoma, Lymphoma, Anal Cancer, Astrocytomas, Atypical Teratoid/Rhabdoid Tumor, Basal Cell Carcinoma of the Skin, Bile Duct Cancer, Bladder Cancer, Bone Cancer, Brain Cancer, Tumors, Breast Cancer, Bronchial Tumors, Carcinoid Tumor, Cardiac (Heart) Tumors, Medulloblastoma, Germ Cell Tumor, Cervical Cancer , Cholangiocarcinoma, Chordoma, Chronic Lymphocytic Leukemia (CLL), Chronic Myelogenous Leukemia (CML), Colorectal Cancer, Craniopharyngioma, Cutaneous T-Cell Lymphoma, Ductal Carcinoma In Situ (DCIS), Embryonal Tumor, Ependymoma, Esophageal Cancer, Esthesioneuroblastoma (Head and Neck Cancer), Ewing Sarcoma (Bone Cancer), Extracranial Germ Cell Tumor, Eye Cancer, Intraocular Melanoma, Retinoblastoma, Fallopian Tube Cancer, Fibrous Histiocytoma of Bone, Osteosarcoma, Gallbladder Cancer, Gastric (Stomach) Cancer, Gastrointestinal Carcinoid Tumor, Gastrointestinal Stromal Tumors (GIST) (Soft Tissue Sarcoma), Extragonadal Germ Cell Tumors, Ovarian Germ Cell Tumors, Testicular Cancer, Gestational Trophoblastic Disease, Hairy Cell Leukemia, Heart Tumors, Histiocytosis, Langerhans Cell Hodgkin Lymphoma, Hypopharyngeal Cancer, Intraocular Melanoma, Islet Cell Tumors, Pancreatic Neuroendocrine Tumors, Kidney (Renal Cell) Cancer, Langerhans Cell Histiocytosis, Laryngeal Cancer, Leukemia, Lip and Oral Cavity Cancer, Liver Cancer, Non-Small Cell Lung Cancer, Small Cell Lung Cancer, Pleuropulmonary Blastoma, Tracheobronchial Tumor, Male Breast Cancer, Malignant Fibrous Histiocytoma of Bone, Melanoma, Intraocular (Eye), Merkel Cell Carcinoma, Mesothelioma, Metastatic Squamous Neck Cancer, Midline Tract Carcinoma, Mouth Cancer, Multiple Endocrine Neoplasia Syndromes, Multiple Myeloma/Plasma Cell Neoplasms, Mycosis Fungoides, Myelodysplastic Syndromes, Myelodysplastic/Myeloproliferative Neoplasms, Myelogenous Leukemia, Chronic (CML) Myeloid Leukemia, Nasal Cavity and Paranasal Sinus Cancer, Nasopharyngeal Cancer, Neuroblastoma, Non-Hodgkin Lymphoma, Oral Cancer, Lip and Oral Cavity Cancer, Oropharyngeal Cancer, Osteosarcoma, Undifferentiated Pleomorphic Sarcoma of Bone, Ovarian Cancer, Pancreatic Cancer, Pancreatic Neuroendocrine Tumors, Papillomatosis, Paraganglioma, Paranasal Sinus and Nasal Cavity, Parathyroid Cancer, Penile Cancer, Pharyngeal Cancer, Pheochromocytoma, Pituitary Tumor, Plasma Cell Neoplasm/Multiple Myeloma, Pleuropulmonary Blastoma, Primary Central Nervous System (CNS) Lymphoma, Primary Peritoneal Cancer, Prostate Cancer, Rectal Cancer, Retinoblastoma, Rhabdomyosarcoma, Salivary Gland Cancer, Ewing Sarcoma (Bone Cancer), Kaposi Sarcoma, Osteosarcoma, Soft Tissue Sarcoma, Uterine Sarcoma, Sézary Syndrome, Skin Cancer, Small Intestine Cancer, Squamous Cell Carcinoma of the Skin, T-Cell Lymphoma, Testicular Cancer, Throat Cancer, Oropharyngeal Cancer, Hypopharyngeal Cancer, Thymoma, Thymic Carcinoma, Thyroid Cancer, Transitional Cell Cancer of the Renal Pelvis and Ureter, Urethral Cancer, Uterine Cancer, Endometrial Cancer, Vaginal Cancer, Vascular Tumor, Vulvar Cancer, Wilms Tumor, or any combination thereof.

6. The method according to any one of claims 3-5 wherein the cancer is Bladder Cancer, Breast Cancer, Colon cancer, Rectal Cancer, Endometrial Cancer, Kidney Cancer, Leukemia, Liver Cancer, Lung Cancer, Melanoma, Non-Hodgkin Lymphoma, Pancreatic Cancer, Prostate Cancer, Thyroid Cancer, or any combination thereof.

7. The method according to any one of claims 3-4 wherein the subject has a tumor.

8. The method according to claim 7 wherein the tumor is Atypical Teratoid/Rhabdoid Tumor, Bronchial Tumors, Carcinoid Tumor, Cardiac (Heart) Tumors, Germ Cell Tumor, Embryonal Tumor, Extracranial Germ Cell Tumor, Gastrointestinal Carcinoid Tumor, Gastrointestinal Stromal Tumors (GIST), Extragonadal Germ Cell Tumors, Ovarian Germ Cell Tumors, Heart Tumors, Islet Cell Tumors, Pancreatic Neuroendocrine Tumors, Tracheobronchial Tumor, Vascular Tumor, Wilms Tumor, or any combination thereof.

9. The method according to any one of claims 1-8 wherein the peripheral blood leukocytes or sentinel lymph node leukocytes comprise a dendritic cell.

10. The method according to any one of claims 1-9 wherein the at least one antigen-specific T cell is a CD8 T cell or a CD4 T cell.

11. The method according to claim 10 wherein the CD4 T cell is a T_{H1}, T_{H2} or T_{H17} T cell.

12. The method according to any one of claims 1-11 further comprising contacting the peripheral blood leukocytes or sentinel lymph node leukocytes of the subject with at least one effector molecule or a second delivery vehicle molecule encapsulating an mRNA encoding an effector molecule to thereby form an mRNA nanoparticle, wherein the effector molecule is a T cell reprogramming molecule, a co-stimulatory molecule, a transcription factor, an antibody or antigen-binding fragment thereof, or a molecule that enhances T cell proliferation.

13. The method according to claim 12 wherein the molecule that enhances T cell proliferation is IL2, IL3, IL4, IL7, IL15, IL18, 4-1BB, CD3z, CD28, an anti-PD1 antibody, or an anti-CTLA4 antibody.

14. The method according to any one of claim 12 or 13 wherein the T cell reprogramming molecule is IL12, IL2, IL7, IL15, IL18, IL21, IL3, IFNα, IFNβ, IFNγ, or TNF-α.

15. The method according to any one of claims 12-14 wherein the co-stimulatory molecule is CD80, CD86, ICOS Ligand, CD70, 4-1BBL, CD40, CD40L, OX40, OX40L, TCF7, ICAM-1, LFA-1, LFA-2, LFA-3, LIGHT, or HVEM.

16. The method according to any one of claims 12-15 wherein the transcription factor is human telomerase, PU.1, CEPBA, CIITA, an HLA, β2 Microglobulin, TAP-1, TAP-2, IRF4, STAT3, or invariant chain Li.

17. The method according to any one of claims 12-16 wherein the antibody or antigen-binding fragment thereof is an anti-CD3 antibody, an anti-CD28 antibody, an anti-CD40 antibody, an anti-OX40 antibody, an anti-PD1 antibody, an anti-CTLA4 antibody, an anti-TIGIT antibody, an anti-LAG3 antibody, an anti-GITR antibody, or an antigen-binding fragment thereof.

18. The method according to any one of claims 1-17 further comprising expanding a number of the at least one antigen-specific T cell.

19. The method according to any one of claims 1-18 wherein the number of the at least one antigen-specific T cell number is passively expanded by exposing the T cells to the delivery vehicle molecule encapsulating the mRNA to form an mRNA nanoparticle, or an antigen-presenting cell that has been exposed to the mRNA nanoparticle, under conditions compatible with cell viability but without providing additional inputs or manipulation.

20. The method according to claim 19 wherein the passively expanded T cell number is further expanded and specialized by exposure to a delivery vehicle molecule encapsulating an mRNA to form an mRNA nanoparticle comprising an mRNA encoding a T cell re-programming molecule, a co-stimulatory molecule, or a transcription factor.

21. The method according to any one of claims 18-20 wherein the workflow is automated and cells are periodically sampled to determine cell number, viability, specificity, phenotype, or any combination thereof, to produce a T cell therapy product.

22. The method according to any one of claims 1-21 wherein the control is a healthy cognate tissue, polynucleotide, polypeptide, or peptide, or is an accepted wild-type sequence of a polynucleotide, polypeptide or peptide.

23. The method according to any one of claims 1-22 wherein the mRNA encodes a polypeptide fused to a hCD1d sorting peptide, thereby increasing antigen presentation.

24. A method comprising administering a therapeutically effective dose of antigen-specific T cells to a patient in need thereof.

25. The method according to claim 24 wherein the antigen-specific T cells are passively expanded in number in an environment compatible with cell viability without adding any additional inputs or manipulation.

26. The method according to any one of claims 24-25 wherein the antigen-specific T cells target tumor cells.

27. The method according to claim 26 wherein the tumor cells are cancerous cells.

28. The method according to any one of claims 24-27 wherein the antigen-specific T cells administered to the patient are syngeneic T cells.

29. The method according to any one of claims 24-28 wherein the antigen-specific T cells administered to the patient are autologous T cells.

30. A method comprising administering a therapeutically effective dose of antigen-specific T cells to an autoimmune patient in need thereof.

31. The method according to claim 30 wherein the autoimmune disease is rheumatoid arthritis, Achalasia, Addison's disease, Adult Still's disease, Agammaglobulinemia, Alopecia areata, Amyloidosis, Ankylosing spondylitis, Anti-GBM/Anti-TBM nephritis, Antiphospholipid syndrome, Autoimmune angioedema, Autoimmune dysautonomia, Autoimmune encephalomyelitis, Autoimmune hepatitis, Autoimmune inner ear disease (AIED), Autoimmune myocarditis, Autoimmune oophoritis, Autoimmune orchitis, Autoimmune pancreatitis, Autoimmune retinopathy, Autoimmune urticarial, Axonal & neuronal neuropathy (AMAN), Baló disease, Behcet's disease, Benign mucosal pemphigoid, Bullous pemphigoid, Castleman disease (CD), Celiac disease, Chagas disease, Chronic inflammatory demyelinating polyneuropathy (CIDP), Chronic recurrent multifocal osteomyelitis (CRMO), Churg-Strauss Syndrome (CSS) or Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Discoid lupus, Dressler's syndrome, Endometriosis, Eosinophilic esophagitis (EoE), Eosinophilic fasciitis, Erythema nodosum, Essential mixed cryoglobulinemia, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenia purpura (ITP), Inclusion body myositis (IBM), Interstitial cystitis (IC), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus erythematosus, Lyme disease, chronic Meniere's disease, Microscopic polyangiitis (MPA), Mixed connective tissue disease (MCTD), Mooren's ulcer, Mucha-Habermann disease, Multifocal Motor Neuropathy (MMN), Multiple sclerosis, Myasthenia gravis, Myositis, Narcolepsy, Neonatal Lupus erythematosus, Neuromyelitis optica, Neutropenia, Ocular cicatricial pemphigoid, Optic neuritis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatic, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Sarcoidosis, Schmidt syndrome, Scleritis, Scleroderma, Sjögren's syndrome, Sperm & testicular autoimmunity, Stiff person syndrome (SPS), Subacute bacterial endocarditis (SBE), Susac's syndrome, Sympathetic ophthalmia (SO), Takayasu's arteritis, Temporal arteritis/Giant cell arteritis, Thrombocytopenia purpura (TTP), Thyroid eye disease (TED), Tolosa-Hunt syndrome (THS), Transverse myelitis, Type 1 diabetes, Ulcerative colitis (UC), Undifferentiated connective tissue disease (UCTD), Uveitis, Vasculitis, Vitiligo, or Vogt-Koyanagi-Harada Disease.

32. The method according to any one of claims 30-31 wherein the antigen-specific T cells administered to the patient are syngeneic T cells.

33. The method according to any one of claims 30-32 wherein the antigen-specific T cells administered to the patient are autologous T cells.

34. A method comprising administering a therapeutically effective dose of antigen-specific T cells to a patient with an infectious disease or an inflammatory disease that is in need thereof.

35. The method according to claim 34 wherein the antigen-specific T cells administered to the patient are syngeneic T cells.

36. The method according to any one of claims 34-35 wherein the antigen-specific T cells administered to the patient are autologous T cells.

37. A method comprising:
contacting peripheral blood leukocytes or sentinel lymph node leukocytes from a subject exposed to an antigenic polypeptide with a delivery vehicle encapsulating an mRNA to form an mRNA nanoparticle comprising an mRNA encoding the antigenic polypeptide or an antigenic fragment thereof;
expanding a number of at least one antigen-specific T cell from the peripheral blood leukocytes or sentinel lymph node leukocytes; and
administering an effective dose of the antigen-specific T cells to the subject, thereby boosting the immune response to the antigenic polypeptide.

38. The method according to claim 37 wherein the subject is exposed to the antigen in the form of a vaccine.

39. The method according to any one of claims 37-38 wherein the antigen is expressed in vivo from an mRNA introduced into the subject.

40. A method comprising administering a delivery vehicle molecule encapsulating an mRNA to form an mRNA nanoparticle vaccine to a subject at risk of having a disease, wherein the mRNA encodes an antigenic polypeptide derived from the subject, thereby vaccinating the subject by inducing an immune response in the subject.

41. The method according to claim 40 further comprising a second delivery of an mRNA nanoparticle vaccine, thereby boosting the immune response.

42. The method according to claim 41 wherein the mRNA of the administered mRNA nanoparticle vaccine and the mRNA of the second delivered mRNA nanoparticle vaccine are identical mRNAs.

43. The method according to claim 41 wherein the administered mRNA nanoparticle vaccine and the second delivered mRNA nanoparticle vaccine are identical mRNA nanoparticle vaccines.

44. The method according to any one of claims 41-43, wherein the delivery vehicle encapsulates the mRNA to form a multicomponent lipitoid mRNA nanoparticle.

45. The method according to any one of claims 41-43 wherein the administered mRNA nanoparticle vaccine comprises a first delivery vehicle molecule, and the second delivered mRNA nanoparticle vaccine comprises a second delivery vehicle molecule, and the first delivery vehicle molecule and the second delivery vehicle molecule are the same.

46. The method according to any one of claims 41-43 wherein the administered mRNA nanoparticle vaccine comprises a first delivery vehicle molecule, and the second delivered mRNA nanoparticle vaccine comprises a second delivery vehicle molecule, and the first delivery vehicle molecule and the second delivery vehicle molecule are different.

47. A vaccine comprising an mRNA nanoparticle comprising an mRNA encoding a peptide comprising a neo-epitope.

48. The vaccine according to claim 47 wherein the mRNA nanoparticle comprises a delivery vehicle molecule that is a multicomponent lipitoid-based nanoparticle.


SEQUENCE LISTING (22 sequences)

SEQ ID NO: 1 - protein, 16 aa, Homo Sapiens
MLFLLLPLLAVLPGDG

SEQ ID NO: 2 - protein, 17 aa, Homo Sapiens
MLLLPFQLLAVLFPGGN

SEQ ID NO: 3 - protein, 17 aa, Homo Sapiens
MLFLQFLLLALLLPGGD

SEQ ID NO: 4 - protein, 19 aa, Homo Sapiens
MGCLLFLLLWALLQAWGSA

SEQ ID NO: 5 - protein, 19 aa, Homo Sapiens
MLLLFLLFEGLCCPGENTA

SEQ ID NO: 6 - protein, 21 aa, Homo Sapiens
MGPWGWKLRWTVALLLAAAGT

SEQ ID NO: 7 - protein, 19 aa, Homo Sapiens
MLTPPLLLLLPLLSALVAA

SEQ ID NO: 8 - protein, 21 aa, Homo Sapiens
GFIILAVIVPLLLLIGLALWF

SEQ ID NO: 9 - protein, 21 aa, Homo Sapiens
IVLAIIVPSLLLLLCLALWYM

SEQ ID NO: 10 - protein, 21 aa, Homo Sapiens
NWIALVVIVPLVILIVLVLWF

SEQ ID NO: 11 - protein, 21 aa, Homo Sapiens
MGLIALAVLACLLFLLIVGFT

SEQ ID NO: 12 - protein, 21 aa, Homo Sapiens
SIFLILICLTVIVTLVILVVV

SEQ ID NO: 13 - protein, 22 aa, Homo Sapiens
ALSIVLPIVLLVFLCLGVFLLW

SEQ ID NO: 14 - protein, 25 aa, Homo Sapiens
HIASILIPLLLLLLLVLVAGVVFWY

SEQ ID NO: 15 - protein, 6 aa, Homo Sapiens
RKRCFC

SEQ ID NO: 16 - protein, 9 aa, Homo Sapiens
RRRSYQNIP

SEQ ID NO: 17 - protein, 10 aa, Homo Sapiens
KKHCSYQDIL

SEQ ID NO: 18 - protein, 13 aa, Homo Sapiens
SRFKRQTSYQGVL

SEQ ID NO: 19 - protein, 63 aa, Homo Sapiens
DSRLKKQSSNKNILSPHTPSPVFLMGANTQDTKNSRHQFCLAQVSWIKNRVLKKWKTRLNQLW

SEQ ID NO: 20 - protein, 50 aa, Homo Sapiens
KNWRLKNINSINFDNPVYQKTTEDEVHICHNQDGYSYPSRQMVSLEDDVA

SEQ ID NO: 21 - protein, 100 aa, Homo Sapiens
KRRVQGAKGFQHQRMTNGAMNVEIGNPTYKMYEGGEPDDVGGLLDADFALDPDKPTNFTNPVYATLYMGGHGSRHSLASTDEKRELLGRGPEDEIGDPLA

SEQ ID NO: 22 - protein, 12 aa, Homo Sapiens
APQALQSYHLAA